(19) United States
(12) Patent Application Publication
(10) Pub. No.: US 2024/0400534 A1
(43) Pub. Date: Dec. 5, 2024

(54) CHEMICAL TOOLS FOR DRUG TARGET IDENTIFICATION AND CHARACTERIZATION

(71) Applicant: Dana-Farber Cancer Institute, Inc., Boston, MA (US)

(72) Inventors: Jarrod MARTO, Wayland, MA (US); Justin KIM, Jamaica Plain, MA (US); Guillaume ADELMANT, Cambridge, MA (US); Dahye KANG, Brookline, MA (US)

(73) Assignee: Dana-Farber Cancer Institute, Inc., Boston, MA (US)

(21) Appl. No.: 18/697,461

(22) PCT Filed: Sep. 30, 2022
(86) PCT No.: PCT/US2022/045371

§ 371 Date: Mar. 30, 2024

Related U.S. Application Data
(60) Provisional application No. 63/320,836, filed on Mar. 17, 2022.
(60) Provisional application No. 63/250,445, filed on Sep. 30, 2021.

(51) Int. Cl. C07D 333/20; C07D 233/88; C12Q 1/37

(52) U.S. Cl. CPC C07D 333/20; C07D 233/88; C12Q 1/37

(57) ABSTRACT

Reagents and methods for the design and analysis of reporter probes optimized for various chemoproteomic assays.

### CROSS-REFERENCE TO RELATED APPLICATIONS

This application is a National Stage Entry of PCT Application No. PCT/US2022/045371, filed on Sep. 30, 2022, which claims benefit of U.S. Provisional Application No. 63/250,445, filed Sep. 30, 2021, and U.S. Provisional Application No. 63/320,836, filed Mar. 17, 2022, each of which are incorporated herein by reference in their entireties.

### GOVERNMENT SUPPORT

This invention was made with government support under grant number DP2 ES030448 awarded by The National Institutes of Health. The government has certain rights in the invention.

### BACKGROUND

Covalent inhibitors have re-emerged as compelling alternatives to reversible, small molecule drugs (Singh et al., Nat. Rev. Drug Discov., 10(4):307-317 (2011); Kalgutkar et al., Expert Opin. Drug Discov., 7(7):561-581 (2012); Lagoutte et al., Curr. Opin. Chem. Biol. 39:54-63 (2007)). Structure-guided design enables improved selectivity through a combination of a moderate-affinity scaffold which positions an electrophilic warhead in proximity to a non-conserved nucleophilic amino acid (e.g., cysteine) on the protein target. Other advantages of covalent inhibitors compared to their reversible counterparts include high efficacy at lower concentration and less frequent dosing, complete target inhibition, with activity restored only after de novo synthesis of new protein, and wider tolerance for pharmacokinetic parameters. FDA-approved covalent drugs span several clinical indications (Singh et al., Nat. Rev. Drug Discov., 10(4):307-317 (2011); Mah et al., Bioorg. Med. Chem. Lett., 24(1):33-39 (2014); De Cesco et al., Eur. J. Med. Chem., 138:96-114 (2017); Bauer, Drug Discov. Today, 20(9):1061-1073 (2015)). The successes of neratinib (targeting HER2/EGFR, breast cancer) (Mundhenke et al., Breast Care (Basel), 4(6):373-378 (2009)), afatinib (targeting EGFR, non-small cell lung cancer, NSCLC) (Engle et al., Am. J. Health Syst. Pharm., 71(22):1933-8193 (2014)), osimertinib (targeting mutant-EGFR, NSCLC) (Cross et al., Cancer Discov., 4(9):1046-1061 (2014)), ibrutinib and acalabrutinib (targeting BTK, chronic lymphocytic leukemia, CLL) (Herman et al., Blood, 117(23):6287-6296 (2011); Byrd et al., N. Engl. J. Med., 374(4):323-332 (2016)) have driven renewed enthusiasm for new covalent drugs for cancer therapy.

The pharmacologic benefits of covalent drugs are tempered by concerns that modification of off-target proteins may result in toxicity. An elegant chemoproteomic study classified over 1,000 cysteine residues in the proteome as ‘hyper-reactive’ (Weerapana et al., Nature, 468(7325):790-795 (2010)), or prone to spurious modification by covalent compounds. In principle, chemoproteomic methods provide a powerful approach to identify potential off-target liabilities by quantifying covalent inhibitor binding across the proteome. However, in practice these approaches are typically reserved for late-stage inhibitors, meaning that off-target liabilities may not be discovered until much later in the development pipeline.

### SUMMARY

A first aspect of the present invention is directed to compounds represented by formulas I and II:

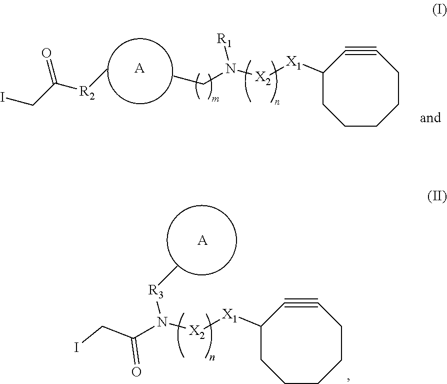

wherein R₁, R₂, R₃, X₁, X₂, A, m, and n are as defined herein, or a pharmaceutically acceptable salt or stereoisomer thereof.

Other aspects of the present invention are directed to compounds represented by formulas III and IV:

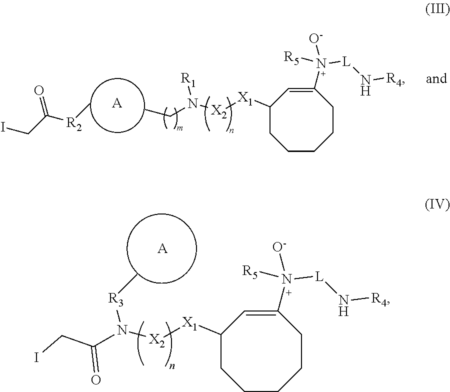

wherein R₁, R₂, R₃, R₄, R₅, X₁, X₂, A, L, m, and n are as defined herein, or a pharmaceutically acceptable salt or stereoisomer thereof.

Further aspects of the present invention are directed to processes of preparing compounds of formulas III and IV. Processes for making compounds of formula III entail reacting a compound of formula I with a compound of formula V,

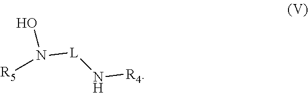

Processes for making compounds of formula IV entail reacting a compound of formula II with a compound of formula V.

Another aspect of the present invention is directed to a composition that includes a compound of formula I-IV or a pharmaceutically acceptable salt or stereoisomer thereof, and a carrier.

Further aspects of the present invention are directed to methods of identifying cysteine residues on a polypeptide that may be targeted by a compound, comprising:
reacting a compound of formula I-IV (“probe”) with a polypeptide, thereby alkylating the polypeptide at cysteine residues therein;
digesting the alkylated polypeptide with at least one proteolytic enzyme, thereby producing probe-labeled peptide fragments of the alkylated polypeptide;
isolating the probe-labeled peptide fragments on a solid phase support;
contacting the thus-isolated probe-labeled peptide fragments with a diboron reagent, thereby releasing/eluting probe-labeled peptide fragments at cysteine residues thereof, and identifying the cysteine residues on the polypeptide.

Another aspect of the present invention is directed to a method of quantifying the number of cysteine residues on a polypeptide that are targeted by a compound, comprising:
(i) reacting a compound of formula I-IV (“probe”) at a fixed concentration with a first mixture comprising one or more polypeptides to form a second mixture comprising one or more compounds of formula I-IV-polypeptide conjugates, wherein each of the compounds of formula I-IV-polypeptide conjugates comprise one or more thioether bonds;
(ii) repeating (i) for a first labelled compound of formula I-IV containing one stable isotope to form a third mixture comprising one or more isotopically labeled compounds of formula I-IV-polypeptide conjugates, wherein each of the isotopically labeled compounds of formula I-IV-polypeptide conjugates comprise one or more thioether bonds;
   a) repeating (ii) for a second labelled compound of formula I-IV containing two stable isotopes to form a fourth mixture comprising one or more isotopically labeled compounds of formula I-IV-polypeptide conjugates, wherein each of the isotopically labeled compounds of formula I-IV-polypeptide conjugates comprise one or more thioether bonds;
   b) repeating (ii) up to 6 more times for each successive compound of formula I-IV containing more than two stable isotopes;
(iii) combining the individual mixtures formed in (i) and (ii) to form a combined mixture;
(iv) enzymatically digesting the combined mixture to form a mixture of peptides comprising a combination of (a) one or more compounds of formula I-IV-polypeptide conjugates and (b) one or more isotopically labeled compounds of formula I-IV-polypeptide conjugates, whereby each conjugate is formed through one or more thioether bonds;
(v) capturing the polypeptide conjugates on a solid phase support;
(vi) contacting the thus-isolated polypeptide conjugates with a diboron reagent, thereby releasing the polypeptides;
(vii) analyzing the polypeptides via a targeted mass spectrometry assay;
(viii) detecting one or more thiolated ions, or derivatives ions thereof produced in the targeted mass spectrometry assay; and
(ix) determining target engagement stoichiometry for the compound of formula I-IV-polypeptide conjugate based on the ratio of thiolated ions, or derivative ions thereof, derived from the isotopically labeled to the unlabeled compounds of formula I-IV-peptide conjugates produced in the targeted mass spectrometry assay.

Another aspect of the present invention is directed to a method of quantifying the number of cysteine residues on a polypeptide that are targeted by a compound, comprising:
(i) reacting a compound of formula I-IV (“probe”) at a fixed concentration with a first mixture comprising one or more polypeptides to form a second mixture comprising one or more compounds of formula I-IV-polypeptide conjugates, wherein each of the compounds of formula I-IV-polypeptide conjugates comprise one or more thioether bonds;
(ii) repeating (i)×3 to form a third, fourth, and fifth mixtures;
(iii) enzymatically digesting the second, third, fourth, and fifth mixtures;
(iv) capturing the polypeptides on a solid phase support;
(v) isotopically labeling the captured polypeptides from the third, fourth, and fifth mixtures;
(vi) combining the individually captured polypeptides to form a combined mixture of captured polypeptides;
(vi) contacting the captured polypeptides with a diboron reagent, thereby releasing the polypeptides;
(vii) analyzing the polypeptides via a targeted mass spectrometry assay;
(viii) detecting one or more thiolated ions, or derivatives ions thereof produced in the targeted mass spectrometry assay; and
(ix) determining target engagement stoichiometry for the compound of formula I-IV-polypeptide conjugate based on the ratio of thiolated ions, or derivative ions thereof, derived from the isotopically labeled to the unlabeled compounds of formula I-IV-peptide conjugates produced in the targeted mass spectrometry assay.

### BRIEF DESCRIPTION OF THE DRAWINGS

FIG. 1 is a schematic depicting the modular assembly of chemoproteomic tools.

FIG. 2A is a schematic showing that thioether bonds exhibit unique tandem mass spectrometry (MS/MS) fragmentation behavior. FIG. 2B illustrates structures of small molecule alkylating scaffolds.

FIG. 3 is an image of capillary electrophoresis-mass spectrometry (CE-MS) demonstrating >95% efficiency at each step with new reagents. Scale of y-axis is the same for extracted ion chromatography shown on upper and lower plots.

FIG. 4 is a Western blot of live K562 cells that were treated with iodoacetamide (IAA) or with iodo-methyl imidazole (IMIA) for indicated times. Additional cultures were treated with DMSO or THZ1 for 6 hours. Cell extracts were treated with THZ1-DTB to label any remaining CDK7-Cys312, followed by streptavidin PD and CDK7 Western blot.

FIG. 5 is a Western blot and target occupancy (TO) assay for THZ1-CDK7. Position of isotopes are indicated by stars overlaying each IMIA 4plex reagent. Red traces show extracted ion chromatograms for thiolated ions detected in high energy MS/MS. Samples were processed in parallel for THZ1-DTB PD and CDK7 Western blot.

FIG. 6 is a schematic showing alkylation and release of cyclooctyne (CO)-caged scaffolds.

FIG. 7A-FIG. 7B show an alternative route for functionalizing reporter scaffolds. FIG. 7A schematically shows different modes of cyclooctyne attachment. FIG. 7B shows that cyclooctynes can be attached at a secondary site on the reporter, should the single attachment point be synthetically inaccessible.

FIG. 8 shows representative reporter scaffolds. Each base scaffold circled in dashed line is modified at the sites marked with an asterisk to optimize the yield of thiolated reporter ions.

FIG. 9 is a schematic showing structurally distinct compounds as chemical bar codes to encode dose-response of different covalent inhibitors or electrophilic fragments and the read out of data generated from a single high-content chemical proteomics assay.

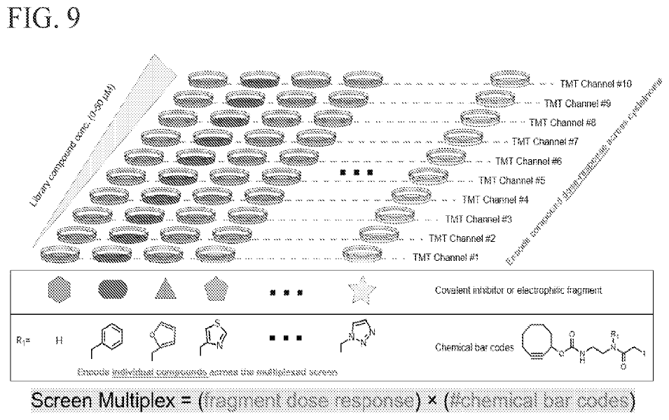

FIG. 10 is a bar graph and heatmap showing the binding activity of a broad covalent kinase inhibitor, a broad covalent DUB inhibitor, and the covalent clinical drug Ibrutinib using the chemoproteomic covalent screen.

### DETAILED DESCRIPTION

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated in order to facilitate the understanding of the present invention.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, and the like.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term “about”.

The transitional term “comprising,” which is synonymous with “including,” “containing,” or “characterized by,” is inclusive or open-ended and does not exclude additional, unrecited elements or method steps. When used in the context of the number of heteroatoms in a heterocyclic structure, it means that the heterocyclic group that that minimum number of heteroatoms. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

With respect to compounds of the present invention, and to the extent the following terms are used herein to further describe them, the following definitions apply.

As used herein, the term “alkyl” refers to a saturated linear or branched-chain monovalent hydrocarbon radical. In one embodiment, the alkyl radical is a C₁-C₁₈ group. In other embodiments, the alkyl radical is a C₀-C₆, C₀-C₅, C₀-C₃, C₁-C₁₂, C₁-C₈, C₁-C₆, C₁-C₅, C₁-C₄ or C₁-C₃ group (wherein C₀ alkyl refers to a bond). Examples of alkyl groups include methyl, ethyl, 1-propyl, 2-propyl, i-propyl, 1-butyl, 2-methyl-1-propyl, 2-butyl, 2-methyl-2-propyl, 1-pentyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, heptyl, octyl, nonyl, decyl, undecyl and dodecyl. In some embodiments, an alkyl group is a C₁-C₃ alkyl group. In some embodiments, an alkyl group is a C₁-C₂ alkyl group, or a methyl group.

As used herein, the term “alkylene” refers to a straight or branched divalent hydrocarbon chain linking the rest of the molecule to a radical group, consisting solely of carbon and hydrogen, containing no unsaturation and having from one to 12 carbon atoms, for example, methylene, ethylene, propylene, n-butylene, and the like. The alkylene chain may be attached to the rest of the molecule through a single bond and to the radical group through a single bond. In some embodiments, the alkylene group contains one to 8 carbon atoms (C₁-C₈ alkylene). In other embodiments, an alkylene group contains one to 5 carbon atoms (C₁-C₅ alkylene). In other embodiments, an alkylene group contains one to 4 carbon atoms (C₁-C₄ alkylene). In other embodiments, an alkylene contains one to three carbon atoms (C₁-C₃ alkylene). In other embodiments, an alkylene group contains one to two carbon atoms (C₁-C₂ alkylene). In other embodiments, an alkylene group contains one carbon atom (C₁ alkylene).

As used herein, the term “alkenyl” refers to a linear or branched-chain monovalent hydrocarbon radical with at least one carbon-carbon double bond. An alkenyl includes radicals having “cis” and “trans” orientations, or alternatively, “E” and “Z” orientations. In one example, the alkenyl radical is a C₂-C₁₈ group. In other embodiments, the alkenyl radical is a C₂-C₁₂, C₂-C₁₀, C₂-C₈, C₂-C₆ or C₂-C₃ group. Examples include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl and hexa-1,3-dienyl.

As used herein, the term “alkynyl” refers to a linear or branched monovalent hydrocarbon radical with at least one carbon-carbon triple bond. In one example, the alkynyl radical is a C₂-C₁₈ group. In other examples, the alkynyl radical is C₂-C₁₂, C₂-C₁₀, C₂-C₈, C₂-C₆ or C₂-C₃. Examples include ethynyl prop-1-ynyl, prop-2-ynyl, but-1-ynyl, but-2-ynyl and but-3-ynyl.

The terms “alkoxyl” or “alkoxy” as used herein refer to an alkyl group, as defined above, having an oxygen radical attached thereto, and which is the point of attachment. Representative alkoxyl groups include methoxy, ethoxy, propyloxy, tert-butoxy and the like. An “ether” is two hydrocarbyl groups covalently linked by an oxygen. Accordingly, the substituent of an alkyl that renders that alkyl an ether is or resembles an alkoxyl, such as can be represented by one of —O-alkyl, —O-alkenyl, and —O-alkynyl.

As used herein, the term “halogen” (or “halo” or “halide”) refers to fluorine, chlorine, bromine, or iodine.

As used herein, the term “cyclic group” broadly refers to any group that used alone or as part of a larger moiety, contains a saturated, partially saturated or aromatic ring system e.g., carbocyclic (cycloalkyl, cycloalkenyl), heterocyclic (heterocycloalkyl, heterocycloalkenyl), aryl and heteroaryl groups. Cyclic groups may have one or more (e.g., fused) ring systems. Thus, for example, a cyclic group can contain one or more carbocyclic, heterocyclic, aryl or heteroaryl groups.

As used herein, the term “carbocyclic” (also “carbocyclyl”) refers to a group that used alone or as part of a larger moiety, contains a saturated, partially unsaturated, or aromatic ring system having 3 to 20 carbon atoms, that is alone or part of a larger moiety (e.g., an alkcarbocyclic group). The term carbocyclyl includes mono-, bi-, tri-, fused, bridged, and spiro-ring systems, and combinations thereof. In one embodiment, carbocyclyl includes 3 to 15 carbon atoms (C₃-C₁₅). In one embodiment, carbocyclyl includes 3 to 12 carbon atoms (C₃-C₁₂). In another embodiment, carbocyclyl includes C₃-C₈, C₃-C₁₀ or C₅-C₁₀. In another embodiment, carbocyclyl, as a monocycle, includes C₃-C₈, C₃-C₆ or C₅-C₆. In some embodiments, carbocyclyl, as a bicycle, includes C₇-C₁₂. In another embodiment, carbocyclyl, as a spiro system, includes C₅-C₁₂. Representative examples of monocyclic carbocyclyls include cyclopropyl, cyclobutyl, cyclopentyl, 1-cyclopent-1-enyl, 1-cyclopent-2-enyl, 1-cyclopent-3-enyl, cyclohexyl, perdeuteriocyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, phenyl, and cyclododecyl; bicyclic carbocyclyls having 7 to 12 ring atoms include [4,3], [4,4], [4,5], [5,5], [5,6] or [6,6] ring systems, such as for example bicyclo[2.2.1]heptane, bicyclo[2.2.2]octane, naphthalene, and bicyclo[3.2.2]nonane. Representative examples of spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocyclyl also includes cycloalkyl rings (e.g., saturated or partially unsaturated mono-, bi-, or spiro-carbocycles). The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

Thus, the term carbocyclic also embraces carbocyclylalkyl groups which as used herein refer to a group of the formula —R^{c}-carbocyclyl where R^{c} is an alkylene chain. The term carbocyclic also embraces carbocyclylalkoxy groups which as used herein refer to a group bonded through an oxygen atom of the formula —O—R^{c}-carbocyclyl where R^{c} is an alkylene chain.

As used herein, the term “aryl” used alone or as part of a larger moiety (e.g., “aralkyl”, wherein the terminal carbon atom on the alkyl group is the point of attachment, e.g., a benzyl group), “aralkoxy” wherein the oxygen atom is the point of attachment, or “aroxyalkyl” wherein the point of attachment is on the aryl group) refers to a group that includes monocyclic, bicyclic or tricyclic, carbon ring system, that includes fused rings, wherein at least one ring in the system is aromatic. In some embodiments, the aralkoxy group is a benzoxy group. The term “aryl” may be used interchangeably with the term “aryl ring”. In one embodiment, aryl includes groups having 6-18 carbon atoms. In another embodiment, aryl includes groups having 6-10 carbon atoms. Examples of aryl groups include phenyl, naphthyl, anthracyl, biphenyl, phenanthrenyl, naphthacenyl, 1,2,3,4-tetrahydronaphthalenyl, 1H-indenyl, 2,3-dihydro-1H-indenyl, naphthyridinyl, and the like, which may be substituted or independently substituted by one or more substituents described herein. A particular aryl is phenyl. In some embodiments, an aryl group includes an aryl ring fused to one or more (e.g., 1, 2 or 3) different cyclic groups (e.g., carbocyclic rings or heterocyclic rings), where the radical or point of attachment is on the aryl ring.

Thus, the term aryl embraces aralkyl groups (e.g., benzyl) which as disclosed above refer to a group of the formula —R^{c}-aryl where R^{c} is an alkylene chain such as methylene or ethylene. In some embodiments, the aralkyl group is an optionally substituted benzyl group. The term aryl also embraces aralkoxy groups which as used herein refer to a group bonded through an oxygen atom of the formula —O—R^{c}-aryl where R^{c} is an alkylene chain such as methylene or ethylene.

As used herein, the term “heterocyclyl” refers to a “carbocyclyl” that used alone or as part of a larger moiety, contains a saturated, partially unsaturated or aromatic ring system, wherein one or more (e.g., 1, 2, 3, or 4) carbon atoms have been replaced with a heteroatom (e.g., 0, N, N(O), S, S(O), or S(O)₂). The term heterocyclyl includes mono-, bi-, tri-, fused, bridged, and spiro-ring systems, and combinations thereof. In some embodiments, a heterocyclyl refers to a 3 to 15 membered heterocyclyl ring system. In some embodiments, a heterocyclyl refers to a 3 to 12 membered heterocyclyl ring system. In some embodiments, a heterocyclyl refers to a saturated ring system, such as a 3 to 12 membered saturated heterocyclyl ring system. In some embodiments, a heterocyclyl refers to a heteroaryl ring system, such as a 5 to 14 membered heteroaryl ring system. The term heterocyclyl also includes C₃-C₈ heterocycloalkyl, which is a saturated or partially unsaturated mono-, bi-, or spiro-ring system containing 3-8 carbons and one or more (1, 2, 3 or 4) heteroatoms.

In some embodiments, a heterocyclyl group includes 3-12 ring atoms and includes monocycles, bicycles, tricycles and spiro ring systems, wherein the ring atoms are carbon, and one to 5 ring atoms is a heteroatom such as nitrogen, sulfur or oxygen. In some embodiments, heterocyclyl includes 3- to 7-membered monocycles having one or more heteroatoms selected from nitrogen, sulfur or oxygen. In some embodiments, heterocyclyl includes 4- to 6-membered monocycles having one or more heteroatoms selected from nitrogen, sulfur or oxygen. In some embodiments, heterocyclyl includes 3-membered monocycles. In some embodiments, heterocyclyl includes 4-membered monocycles. In some embodiments, heterocyclyl includes 5-6 membered monocycles. In some embodiments, the heterocyclyl group includes 0 to 3 double bonds. In any of the foregoing embodiments, heterocyclyl includes 1, 2, 3 or 4 heteroatoms. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]⁺OH⁻). Representative examples of heterocyclyls include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydropyranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, thiophenyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls containing a sulfur or oxygen atom and one to three nitrogen atoms are thiazolyl, including thiazol-2-yl and thiazol-2-yl N-oxide, thiadiazolyl, including 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Example 5-membered ring heterocyclyls containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Representative examples of benzo-fused 5-membered heterocyclyls are benzoxazol-2-yl, benzthiazol-2-yl and benzimidazol-2-yl. Example 6-membered heterocyclyls contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl. The pyridine N-oxides and pyridazine N-oxides and the pyridyl, pyrimid-2-yl, pyrimid-4-yl, pyridazinyl and the 1,3,4-triazin-2-yl groups, are yet other examples of heterocyclyl groups. In some embodiments, a heterocyclic group includes a heterocyclic ring fused to one or more (e.g., 1, 2 or 3) different cyclic groups (e.g., carbocyclic rings or heterocyclic rings), where the radical or point of attachment is on the heterocyclic ring, and in some embodiments wherein the point of attachment is a heteroatom contained in the heterocyclic ring.

Thus, the term heterocyclic embraces N-heterocyclyl groups which as used herein refer to a heterocyclyl group containing at least one nitrogen and where the point of attachment of the heterocyclyl group to the rest of the molecule is through a nitrogen atom in the heterocyclyl group. Representative examples of N-heterocyclyl groups include 1-morpholinyl, 1-piperidinyl, 1-piperazinyl, 1-pyrrolidinyl, pyrazolidinyl, imidazolinyl and imidazolidinyl. The term heterocyclic also embraces C-heterocyclyl groups which as used herein refer to a heterocyclyl group containing at least one heteroatom and where the point of attachment of the heterocyclyl group to the rest of the molecule is through a carbon atom in the heterocyclyl group. Representative examples of C-heterocyclyl radicals include 2-morpholinyl, 2- or 3- or 4-piperidinyl, 2-piperazinyl, and 2- or 3-pyrrolidinyl. The term heterocyclic also embraces heterocyclylalkyl groups which as disclosed above refer to a group of the formula —R^{c}-heterocyclyl where R^{c} is an alkylene chain. The term heterocyclic also embraces heterocyclylalkoxy groups which as used herein refer to a radical bonded through an oxygen atom of the formula —O—R^{c}-heterocyclyl where R^{c} is an alkylene chain.

As used herein, the term “heteroaryl” used alone or as part of a larger moiety (e.g., “heteroarylalkyl” (also “heteroaralkyl”), or “heteroarylalkoxy” (also “heteroaralkoxy”), refers to a monocyclic, bicyclic or tricyclic ring system having 5 to 14 ring atoms, wherein at least one ring is aromatic and contains at least one heteroatom. In one embodiment, heteroaryl includes 5-6 membered monocyclic aromatic groups where one or more ring atoms is nitrogen, sulfur or oxygen. Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, imidazopyridyl, pyrazinyl, pyridazinyl, triazinyl, tetrazinyl, tetrazolo[1,5-b]pyridazinyl, purinyl, deazapurinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, 1,2,3-triazol-5-yl, and pyrid-2-yl N-oxide. The term “heteroaryl” also includes groups in which a heteroaryl is fused to one or more cyclic (e.g., carbocyclyl, or heterocyclyl) rings, where the radical or point of attachment is on the heteroaryl ring. Nonlimiting examples include indolyl, indolizinyl, isoindolyl, benzothienyl, benzothiophenyl, methylenedioxyphenyl, benzofuranyl, dibenzofuranyl, indazolyl, benzimidazolyl, benzodioxazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl and pyrido[2,3-b]-1,4-oxazin-3(4H)-one. A heteroaryl group may be mono-, bi- or tri-cyclic. In some embodiments, a heteroaryl group includes a heteroaryl ring fused to one or more (e.g., 1, 2 or 3) different cyclic groups (e.g., carbocyclic rings or heterocyclic rings), where the radical or point of attachment is on the heteroaryl ring, and in some embodiments wherein the point of attachment is a heteroatom contained in the heterocyclic ring.

Thus, the term heteroaryl embraces N-heteroaryl groups which as used herein refer to a heteroaryl group as defined above containing at least one nitrogen and where the point of attachment of the heteroaryl group to the rest of the molecule is through a nitrogen atom in the heteroaryl group. The term heteroaryl also embraces C-heteroaryl groups which as used herein refer to a heteroaryl group as defined above and where the point of attachment of the heteroaryl group to the rest of the molecule is through a carbon atom in the heteroaryl group. The term heteroaryl also embraces heteroarylalkyl groups which as disclosed above refer to a group of the formula —R^{c}-heteroaryl, wherein R^{c} is an alkylene chain as defined above. The term heteroaryl also embraces heteroaralkoxy (or heteroarylalkoxy) groups which as used herein refer to a group bonded through an oxygen atom of the formula —O—R^{c}-heteroaryl, where R^{c} is an alkylene group as defined above.

As used herein, the term “arene” refers to a bivalent aryl radical which may be optionally substituted.

As used herein, the term “heterocyclene” refers to a bivalent heterocyclyl radical which may be optionally substituted.

As used herein, the term “heteroarylene” refers to a bivalent heteroaryl radical which may be optionally substituted.

Unless stated otherwise, and to the extent not further defined for any particular group(s), any of the groups described herein may be substituted or unsubstituted. As used herein, the term “substituted” broadly refers to all permissible substituents with the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc. Representative substituents include halogens, hydroxyl groups, and any other organic groupings containing any number of carbon atoms, e.g., 1-14 carbon atoms, and which may include one or more (e.g., 1, 2, 3, or 4) heteroatoms such as oxygen, sulfur, and nitrogen grouped in a linear, branched, or cyclic structural format.

To the extent not disclosed otherwise for any particular group(s), representative examples of substituents may include alkyl, substituted alkyl (e.g., C₁-C₆, C₁-C₅, C₁-C₄, C₁-C₃, C₁-C₂, C₁), alkoxy (e.g., C₁-C₆, C₁-C₅, C₁-C₄, C₁-C₃, C₁-C₂, C₁), substituted alkoxy (e.g., C₁-C₆, C₁-C₅, C₁-C₄, C₁-C₃, C₁-C₂, C₁), haloalkyl (e.g., CF₃), alkenyl (e.g., C₂-C₆, C₂-C₅, C₂-C₄, C₂-C₃, C₂), substituted alkenyl (e.g., C₂-C₆, C₂-C₅, C₂-C₄, C₂-C₃, C₂), alkynyl (e.g., C₂-C₆, C₂-C₅, C₂-C₄, C₂-C₃, C₂), substituted alkynyl (e.g., C₂-C₆, C₂-C₅, C₂-C₄, C₂-C₃, C₂), cyclic (e.g., C₃-C₁₂, C₅-C₆), substituted cyclic (e.g., C₃-C₁₂, C₅-C₆), carbocyclic (e.g., C₃-C₁₂, C₅-C₆), substituted carbocyclic (e.g., C₃-C₁₂, C₅-C₆), heterocyclic (e.g., C₃-C₁₂, C₅-C₆), substituted heterocyclic (e.g., C₃-C₁₂, C₅-C₆), aryl (e.g., benzyl and phenyl), substituted aryl (e.g., substituted benzyl or phenyl), heteroaryl (e.g., pyridyl or pyrimidyl), substituted heteroaryl (e.g., substituted pyridyl or pyrimidyl), aralkyl (e.g., benzyl), substituted aralkyl (e.g., substituted benzyl), halo, hydroxyl, aryloxy (e.g., C₆-C₁₂, C₆), substituted aryloxy (e.g., C₆-C₁₂, C₆), alkylthio (e.g., C₁-C₆), substituted alkylthio (e.g., C₁-C₆), arylthio (e.g., C₆-C₁₂, C₆), substituted arylthio (e.g., C₆-C₁₂, C₆), cyano, carbonyl, substituted carbonyl, carboxyl, substituted carboxyl, amino, substituted amino, amido, substituted amido, thio, substituted thio, sulfinyl, substituted sulfinyl, sulfonyl, substituted sulfonyl, sulfinamide, substituted sulfinamide, sulfonamide, substituted sulfonamide, urea, substituted urea, carbamate, substituted carbamate, amino acid, and peptide groups.

As used herein, the term “electron donating group” refers to an atom or functional group that releases electron density to neighboring atoms from itself, usually by resonance or inductive effects.

As used herein, the term “electron withdrawing group” refers to an atom or functional group that draws electron density from neighboring atoms to itself, usually by resonance or inductive effects.

As used herein, the term “ionizable group” refers to any uncharged group in a molecular entity that is capable of dissociating by yielding an ion (usually an H⁺ ion) or an electron and itself becoming oppositely charged.

As used herein, the term “small molecule” refers to a molecule, whether naturally-occurring or artificially created (e.g., via chemical synthesis) that has a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.).

In one aspect, compounds of the invention are represented by formula I or II:

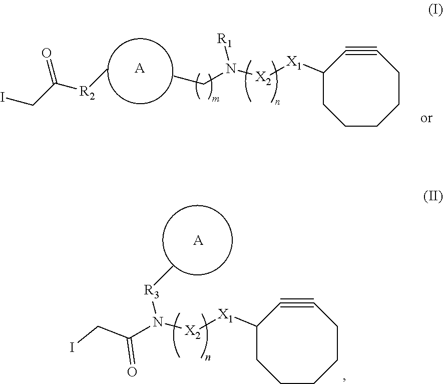

wherein,
X₁ is NR₁, O, S, S(O), or S(O)₂;
each X₂ is independently C(R₁)₂, NR₁, O, C(O), C₆-C₁₀ aryl, or —OCH₂CH₂—;
each R₁ is independently hydrogen, C₁-C₆ alkyl, C₆-C₁₀ aryl, or 5- to 10-membered heteroaryl, wherein said alkyl, aryl, or heteroaryl is optionally substituted;
R₂ is absent or NH;
R₃ is absent, C(O), or C₁-C₃ alkylene;
A is C₁-C₆ alkyl, C₆-C₁₀ aryl, C₆-C₁₀ arene, 5- to 10-membered heteroaryl, 5- to 10-membered heteroarylene, or a small molecule, wherein said alkyl, aryl, or heteroaryl is optionally substituted;
m is an integer from 0-5; and
n is an integer from 0-10;
or a pharmaceutically acceptable salt or stereoisomer thereof.

In some embodiments, X₁ is O. In some embodiments, X₁ is S. In some embodiments, X₁ is S(O) or S(O)₂. In some embodiments, X₁ is NR₁ and R₁ is H.

In some embodiments, each X₂ is independently C(R₁)₂, NR₁, O, C(O), or —OCH₂CH₂—. In some embodiments, each X₂ is independently CHR₁, CH₂, NR₁, O, or C(O). In some embodiments, each X₂ is independently CHR₁, CH₂, NR₁, C(O), or —OCH₂CH₂—. In some embodiments, each X₂ is independently CH₂, NR₁, or C(O). In some embodiments, each X₂ is independently NR₁, C(O), or —OCH₂CH₂—. In some embodiments, each X₂ is independently CH₂, NR₁, or C(O).

In some embodiments, R₃ is absent. In some embodiments, R₃ is C₂ alkylene.

In some embodiments, n is 0. In some embodiments, n is 1. In some embodiments, n is 2. In some embodiments, n is 3. In some embodiments, n is 4. In some embodiments, n is 5.

In some embodiments, m is 0. In some embodiments, m is 1. In some embodiments, m is 2. In some embodiments, m is 3. In some embodiments, m is 5.

In some embodiments, each carbon, nitrogen, and oxygen of the compound of formula I or II is substituted with a stable isotope thereof, wherein the isotope is selected from ¹³C, ¹⁵N, and ¹⁸O.

In some embodiments, the compound of formula I or II contains 1-8 isotopes. In some embodiments, the compound of formula I or II contains 1-4 isotopes. In some embodiments, the compound of formula I or II contains 3 isotopes. In some embodiments, the compound of formula I or II contains 2 isotopes. In some embodiments, the isotope is ²H, ¹³C, ¹⁵N, or ¹⁸O, or a combination of two or more thereof. Possible sites of the compound of formula I or II that can contain isotope(s) are indicated by enclosure in dashed line:

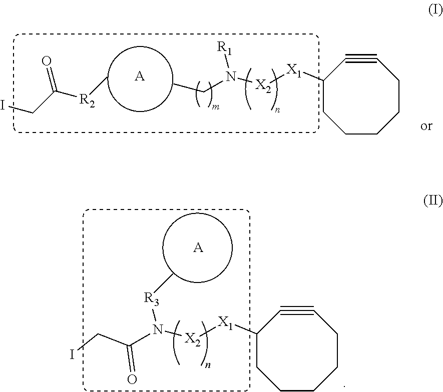

In some embodiments, A is imidazolyl, thiazolyl, furanyl, pyridinyl, triazolyl, or phenyl, and wherein A is optionally substituted.

In some embodiments, A is substituted with one or more electron donating groups. In some embodiments, the electron donating group is —OH, C₁-C₆ alkyl, or C₁-C₆ alkoxyl.

In some embodiments, A is substituted with one or more electron withdrawing groups.

In some embodiments, the electron withdrawing group is —CN, —COOH, or NO₂.

In some embodiments, A is substituted with one or more ionizable groups. In some embodiments, the ionizable group is NH₂.

In some embodiments, A is a small molecule. In certain embodiments, the molecular weight of the small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of the small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol.

In some embodiments, A is an optionally substituted C₃-C₁₂ carbocyclyl.

In some embodiments, A is an optionally substituted C₆-C₁₄ aryl.

In some embodiments, A is an optionally substituted C₆-C₁₄ arene

In some embodiments, A is an optionally substituted 3- to 10-membered heterocyclyl.

In some embodiments, A is an optionally substituted 3- to 10-membered heterocyclene.

In some embodiments, A is an optionally substituted 5- to 10-membered heteroaryl.

In some embodiments, A is an optionally substituted 5- to 10-membered heteroarylene.

In some embodiments, A contains one or more substituents and each substituent for a compound of formula (I or II) is independently alkyl, alkenyl, alkynyl, halo, haloalkyl, cycloalkyl, heterocycloalkyl, hydroxy, alkoxy, cycloalkoxy, heterocycloalkoxy, haloalkoxy, aryloxy, heteroaryloxy, aralkyloxy, alkyenyloxy, alkynyloxy, amino, alkylamino, cycloalkylamino, heterocycloalkylamino, arylamino, heteroarylamino, aralkylamino, N-alkyl-N-arylamino, N-alkyl-N-heteroarylamino, N-alkyl-N-aralkylamino, hydroxyalkyl, aminoalkyl, alkylthio, haloalkylthio, alkylsulfonyl, haloalkylsulfonyl, cycloalkylsulfonyl, heterocycloalkylsulfonyl, arylsulfonyl, heteroarylsulfonyl, aminosulfonyl, alkylaminosulfonyl, cycloalkylaminosulfonyl, heterocycloalkylaminosulfonyl, arylaminosulfonyl, heteroarylaminosulfonyl, N-alkyl-N-arylaminosulfonyl, N-alkyl-N-heteroarylaminosulfonyl, formyl, alkylcarbonyl, haloalkylcarbonyl, alkenylcarbonyl, alkynylcarbonyl, carboxy, alkoxycarbonyl, alkylcarbonyloxy, amino, alkylsulfonylamino, haloalkylsulfonylamino, cycloalkylsulfonylamino, heterocycloalkylsulfonylamino, arylsulfonylamino, heteroarylsulfonylamino, aralkylsulfonylamino, alkylcarbonylamino, haloalkylcarbonylamino, cycloalkylcarbonylamino, heterocycloalkylcarbonylamino, arylcarbonylamino, heteroarylcarbonylamino, aralkylsulfonylamino, aminocarbonyl, alkylaminocarbonyl, cycloalkylaminocarbonyl, heterocycloalkylaminocarbonyl, arylaminocarbonyl, heteroarylaminocarbonyl, N-alkyl-N-arylaminocarbonyl, N-alkyl-N-heteroarylaminocarbonyl, cyano, nitro, or azido.

Representative examples of compounds of formula II include:

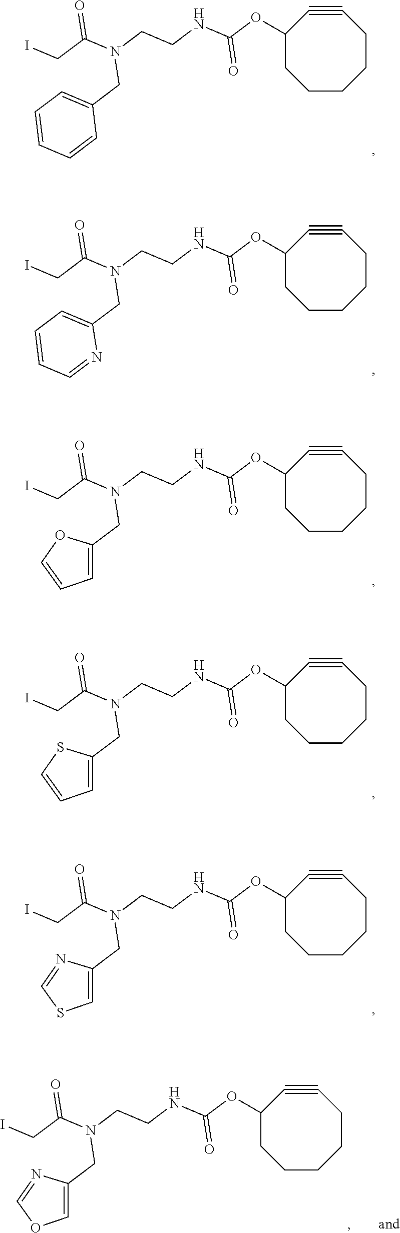 [SMILES: O=C(NCCN(Cc1ccccc1)C(=O)CI)OC1C#CCCCCC1 ; O=C(NCCN(Cc1ccccn1)C(=O)CI)OC1C#CCCCCC1 ; O=C(NCCN(Cc1ccco1)C(=O)CI)OC1C#CCCCCC1 ; O=C(NCCN(Cc1cccs1)C(=O)CI)OC1C#CCCCCC1 ; O=C(NCCN(Cc1cscn1)C(=O)CI)OC1C#CCCCCC1 ; O=C(NCCN(Cc1cocn1)C(=O)CI)OC1C#CCCCCC1]

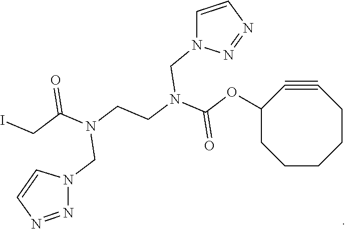 [SMILES: O=C(CI)N(CCN(Cn1ccnn1)C(=O)OC1C#CCCCCC1)Cn1ccnn1]

Other inventive compounds of the invention are represented by formulas III or IV:

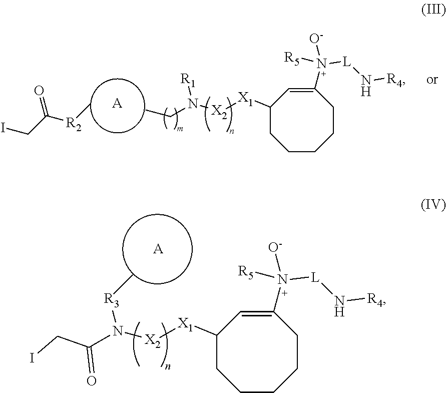

wherein,
X₁ is NR₁, O, S, S(O), or S(O)₂;
each X₂ is independently C(R₁)₂, NR₁, O, C(O), C₆-C₁₀ aryl, or —OCH₂CH₂—;
each R₁ is independently hydrogen, C₁-C₆ alkyl, C₆-C₁₀ aryl, or 5- to 10-membered heteroaryl,
wherein said alkyl, aryl, or heteroaryl is optionally substituted;
R₂ is absent or NH;
R₃ is absent, C(O), or C₁-C₃ alkylene;
R₄ is an affinity handle, a bead, or a combination of these;
R₅ is C₁-C₆ alkyl;
L is an alkylene chain or a PEG chain;
A is C₁-C₆ alkyl, C₆-C₁₀ aryl, C₆-C₁₀ arene, 5- to 10-membered heteroaryl, 5- to 10-membered heteroarylene, or a small molecule, wherein said alkyl, aryl, or heteroaryl is optionally substituted;
m is an integer from 0-5; and
n is an integer from 0-10,
or a pharmaceutically acceptable salt or stereoisomer thereof.

In some embodiments, X₁ is O. In some embodiments, X₁ is S. In some embodiments, X₁ is S(O) or S(O)₂. In some embodiments, X₁ is NR₁ and R₁ is H.

In some embodiments, each X₂ is independently C(R₁)₂, NR₁, O, C(O), or —OCH₂CH₂—. In some embodiments, each X₂ is independently CHR₁, CH₂, NR₁, O, or C(O). In some embodiments, each X₂ is independently CHR₁, CH₂, NR₁, C(O), or —OCH₂CH₂—. In some embodiments, each X₂ is independently CH₂, NR₁, or C(O). In some embodiments, each X₂ is independently NR₁, C(O), or —OCH₂CH₂—. In some embodiments, each X₂ is independently CH₂, NR₁, or C(O).

In some embodiments, R₃ is absent. In some embodiments, R₃ is C₂ alkylene.

In some embodiments, n is 0. In some embodiments, n is 1. In some embodiments, n is 2. In some embodiments, n is 3. In some embodiments, n is 4. In some embodiments, n is 5.

In some embodiments, m is 0. In some embodiments, m is 1. In some embodiments, m is 2. In some embodiments, m is 3. In some embodiments, m is 5.

In some embodiments, R₄ is an affinity handle. The term “affinity handle” refers to a portion of a compound that targets it to an appropriate site of action, e.g., a targeted polypeptide. Representative examples of affinity handles that may be useful include small chemical compounds (such as biotin and derivatives thereof, e.g., desthiobiotin), amino acid (e.g., His and Leu) tags, typically ranging 2 to 20 amino acids in length, and in some embodiments, from 4 to 12 amino acids in length, such as the (His)₆ tag, (His)₄ tag, (His)₃ tag, (His)₂ tag, (Leu)₄ tag, (Leu)₃ tag, (Leu)₂ tag, human influenza hemagglutinin (HA) tag, FLAG® tag, vesicular stomatitis virus glycoprotein (VSV-G) tag, herpes simplex virus (HSV) tag, and V5 tag, human O⁶-alkylguanine-DNA-alkyltransferase (hAGT), chitin binding protein (CBP), maltose binding protein (MBP), Strep-tag, glutathione-S-transferase (GST), SNAP-tag, and CLIP-tag.

In some embodiments, the affinity handle is chloroalkane (HaloTag).

In some embodiments, the affinity handle is biotin or a biotin derivative. Biotin and its derivatives have been widely used as molecular labels in the biotechnology industry for many years. Biotin derivatives that may be suitable for use in the present invention are disclosed in U.S. Pat. No. 8,318,696 and U.S. Patent Application Publication No. 2007/0020206, each of which is incorporated by reference.

In some embodiments, the affinity handle is a protein. In some embodiments, the protein is SNAP-tag or CLIP-tag.

Biotin and its derivatives have been widely used as molecular labels in the biotechnology industry for many years. Biotin derivatives that may be suitable for use in the present invention are disclosed in U.S. Pat. No. 8,318,696 and U.S. Patent Application Publication No. 2007/0020206, each of which is incorporated by reference.

In some embodiments, R₄ is a bead. In some embodiments, the bead is a magnetic bead, polystyrene bead, or agarose bead.

In some embodiments, each carbon, nitrogen, and oxygen of the compound of formula III or IV is substituted with a stable isotope thereof, wherein the isotope is selected from ¹³C, ¹⁵N, and ¹⁸O.

In some embodiments, the compound of formula III or IV contains 1-8 isotopes. In some embodiments, the compound of formula III or IV contains 1-4 isotopes. In some embodiments, the compound of formula III or IV contains 3 isotopes. In some embodiments, the compound of formula III or IV contains 2 isotopes. In some embodiments, the isotope is ²H, ¹³C, ¹⁵N, or ¹⁸O, or a combination of two or more thereof. Possible sites of the compound of formula III or IV that can contain isotope(s) are indicated by enclosure in dashed line:

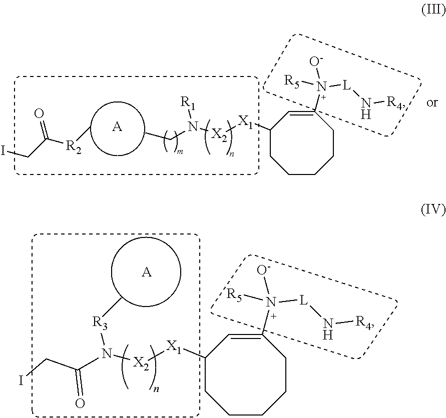

In some embodiments, A is imidazolyl, thiazolyl, furanyl, pyridinyl, triazolyl, or phenyl, and wherein A is optionally substituted.

In some embodiments, A is substituted with one or more electron donating groups. In some embodiments, the electron donating group is —OH, C₁-C₆ alkyl, or C₁-C₆ alkoxyl.

In some embodiments, A is substituted with one or more electron withdrawing groups.

In some embodiments, the electron withdrawing group is —CN, —COOH, or NO₂.

In some embodiments, A is substituted with one or more ionizable groups. In some embodiments, the ionizable group is NH₂.

In some embodiments, A is a small molecule. In certain embodiments, the molecular weight of the small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of the small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol.

In some embodiments, A is an optionally substituted C₃-C₁₂ carbocyclyl.

In some embodiments, A is an optionally substituted C₆-C₁₄ aryl.

In some embodiments, A is an optionally substituted 3- to 10-membered heterocyclyl.

In some embodiments, A is an optionally substituted 5- to 10-membered heteroaryl.

In some embodiments, A contains one or more substituents and each substituent for a compound of formula (III or IV) is independently selected from the group comprising of alkyl, alkenyl, alkynyl, halo, haloalkyl, cycloalkyl, heterocycloalkyl, hydroxy, alkoxy, cycloalkoxy, heterocycloalkoxy, haloalkoxy, aryloxy, heteroaryloxy, aralkyloxy, alkyenyloxy, alkynyloxy, amino, alkylamino, cycloalkylamino, heterocycloalkylamino, arylamino, heteroarylamino, aralkylamino, N-alkyl-N-arylamino, N-alkyl-N-heteroarylamino, N-alkyl-N-aralkylamino, hydroxyalkyl, aminoalkyl, alkylthio, haloalkylthio, alkylsulfonyl, haloalkylsulfonyl, cycloalkylsulfonyl, heterocycloalkylsulfonyl, arylsulfonyl, heteroarylsulfonyl, aminosulfonyl, alkylaminosulfonyl, cycloalkylaminosulfonyl, heterocycloalkylaminosulfonyl, arylaminosulfonyl, heteroarylaminosulfonyl, N-alkyl-N-arylaminosulfonyl, N-alkyl-N-heteroarylaminosulfonyl, formyl, alkylcarbonyl, haloalkylcarbonyl, alkenylcarbonyl, alkynylcarbonyl, carboxy, alkoxycarbonyl, alkylcarbonyloxy, amino, alkylsulfonylamino, haloalkylsulfonylamino, cycloalkylsulfonylamino, heterocycloalkylsulfonylamino, arylsulfonylamino, heteroarylsulfonylamino, aralkylsulfonylamino, alkylcarbonylamino, haloalkylcarbonylamino, cycloalkylcarbonylamino, heterocycloalkylcarbonylamino, arylcarbonylamino, heteroarylcarbonylamino, aralkylsulfonylamino, aminocarbonyl, alkylaminocarbonyl, cycloalkylaminocarbonyl, heterocycloalkylaminocarbonyl, arylaminocarbonyl, heteroarylaminocarbonyl, N-alkyl-N-arylaminocarbonyl, N-alkyl-N-heteroarylaminocarbonyl, cyano, nitro, and azido.

Compounds of the present invention may be in the form of a free acid or free base, or a pharmaceutically acceptable salt. As used herein, the term “pharmaceutically acceptable” in the context of a salt refers to a salt of the compound that does not abrogate the biological activity or properties of the compound, and is relatively non-toxic, i.e., the compound in salt form may be administered to a subject without causing undesirable biological effects (such as dizziness or gastric upset) or interacting in a deleterious manner with any of the other components of the composition in which it is contained. The term “pharmaceutically acceptable salt” refers to a product obtained by reaction of the compound of the present invention with a suitable acid or a base. Examples of pharmaceutically acceptable salts of the compounds of this invention include those derived from suitable inorganic bases such as Li, Na, K, Ca, Mg, Fe, Cu, Al, Zn and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain compounds of the invention can form pharmaceutically acceptable salts with various organic bases such as lysine, arginine, guanidine, diethanolamine or metformin. Suitable base salts include aluminum, calcium, lithium, magnesium, potassium, sodium, or zinc salts.

Compounds of the present invention may have at least one chiral center and thus may be in the form of a stereoisomer, which as used herein, embraces all isomers of individual compounds that differ only in the orientation of their atoms in space. The term stereoisomer includes mirror image isomers (enantiomers which include the (R—) or (S—) configurations of the compounds), mixtures of mirror image isomers (physical mixtures of the enantiomers, and racemates or racemic mixtures) of compounds, geometric (cis/trans or E/Z, R/S) isomers of compounds and isomers of compounds with more than one chiral center that are not mirror images of one another (diastereoisomers). The chiral centers of the compounds may undergo epimerization in vivo; thus, for these compounds, administration of the compound in its (R—) form is considered equivalent to administration of the compound in its (S—) form. Accordingly, the compounds of the present invention may be made and used in the form of individual isomers and substantially free of other isomers, or in the form of a mixture of various isomers, e.g., racemic mixtures of stereoisomers.

In some embodiments, the compound is an isotopic derivative in that it has at least one desired isotopic substitution of an atom, at an amount above the natural abundance of the isotope, i.e., enriched. In one embodiment, the compound includes deuterium or multiple deuterium atoms. Substitution with heavier isotopes such as deuterium, i.e. ²H, may afford certain therapeutic advantages resulting from greater metabolic stability, for example, increased in vivo half-life or reduced dosage requirements, and thus may be advantageous in some circumstances.

The compounds of the present invention may be prepared by crystallization under different conditions and may exist as one or a combination of polymorphs of the compound. For example, different polymorphs may be identified and/or prepared using different solvents, or different mixtures of solvents for recrystallization, by performing crystallizations at different temperatures, or by using various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffractogram and/or other known techniques.

In some embodiments, the pharmaceutical composition comprises a co-crystal of an inventive compound. The term “co-crystal”, as used herein, refers to a stoichiometric multi-component system comprising a compound of the invention and a co-crystal former wherein the compound of the invention and the co-crystal former are connected by non-covalent interactions. The term “co-crystal former”, as used herein, refers to compounds which can form intermolecular interactions with a compound of the invention and co-crystallize with it. Representative examples of co-crystal formers include benzoic acid, succinic acid, fumaric acid, glutaric acid, trans-cinnamic acid, 2,5-dihydroxybenzoic acid, glycolic acid, trans-2-hexanoic acid, 2-hydroxycaproic acid, lactic acid, sorbic acid, tartaric acid, ferulic acid, suberic acid, picolinic acid, salicyclic acid, maleic acid, saccharin, 4,4′-bipyridine p-aminosalicyclic acid, nicotinamide, urea, isonicotinamide, methyl-4-hydroxybenzoate, adipic acid, terephthalic acid, resorcinol, pyrogallol, phloroglucinol, hydroxyquinol, isoniazid, theophylline, adenine, theobromine, phenacetin, phenazone, etofylline, and phenobarbital.

### Methods of Synthesis

In another aspect, the present invention is directed to a method for making an inventive compound, or a pharmaceutically acceptable salt or stereoisomer thereof. Broadly, the inventive compounds and their pharmaceutically acceptable salts and stereoisomers may be prepared by any process known to be applicable to the preparation of chemically related compounds. The compounds of the present invention will be better understood in connection with the synthetic schemes that are described in various working examples and which illustrate non-limiting methods by which the compounds may be prepared.

In another aspect, the present invention is directed to methods for preparing compounds of formula III:

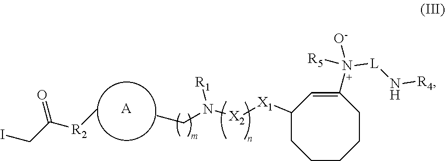

comprising reacting a compound of formula I:

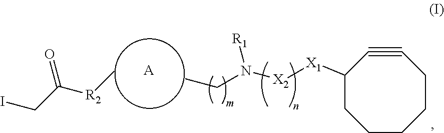

with a compound of formula V:

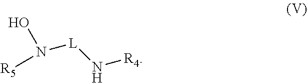

In another aspect, the present invention is directed to methods for preparing compounds of formula IV:

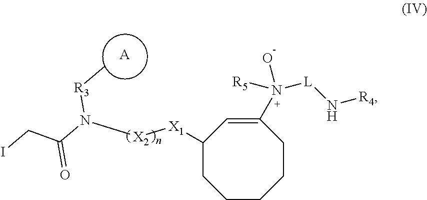

comprising reacting a compound of formula II:

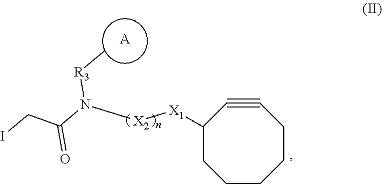

with a compound of formula V:

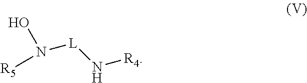

In some embodiments, the reacting is carried out in the presence of a solvent.

In some embodiments, the solvent is an aprotic solvent. In some embodiments, the aprotic solvent is DCM, CHCl₃, CCl₄, DCE, toluene, MeCN, or THF.

In some embodiments, the solvent is a protic solvent. In some embodiments, the protic solvent is MeOH, EtOH, iPrOH, nBuOH, TFE, or HFIP.

In some embodiments, the solvent is a solvent mixture. In some embodiments, the solvent mixture is a mixture of an aprotic solvent and a protic solvent.

In some embodiments, the reaction is carried out in the presence of an aqueous buffer.

In some embodiments, the aqueous buffer is an acidic buffer. In some embodiments, the aqueous buffer is an alkaline buffer.

In some embodiments, the reaction is carried out in the presence of a biological fluid.

In some embodiments, the biological fluid is blood, synovial fluid, lymph, or vitreous fluid.

In some embodiments, the reaction is carried out in the presence of an aqueous solution with biological components such as cell lysate, proteins, nucleic acids, or lipids.

In some embodiments, the reaction is carried out at a temperature from about 0° C. to 70° C. In some embodiments, the reacting is carried out at a temperature is about 20° C.-25° C.

In some embodiments, the reaction is carried out over a week. In some embodiments, the reaction is carried out over five days. In some embodiments, the reaction is carried out over three days. In some embodiments, the reaction is carried out over a period of 24 hours. In some embodiments, the reaction is carried out over a period of 18 hours. In some embodiments, the reaction is carried out over a period of 12 hours. In some embodiments, the reaction is carried out over a period of 6 hours. In some embodiments, the reaction is carried out over a period of 3 hours. In some embodiments, the reaction is carried out over a period of 2 hours. In some embodiments, the reaction is carried out over a period of 1 hour. In some embodiments, the reaction is carried out over a period of 45 minutes. In some embodiments, the reaction is carried out over a period of 30 minutes. In some embodiments, the reaction is carried out over a period of 15 minutes. In some embodiments, the reaction is carried out over a period of 5 minutes. In some embodiments, the reaction is carried out over a period of 1 minute.

### Compositions

Another aspect of the present invention is directed to a composition that includes inventive compound or a pharmaceutically acceptable salt or stereoisomer thereof, and a carrier. The term “carrier,” as known in the art, refers to a material, composition or vehicle, suitable for administering compounds of the present invention to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. Depending on the type of formulation, the composition may also include one or more excipients.

### Methods of Use

In some aspects, the present invention is directed to methods of identifying cysteine residues on a polypeptide that may be targeted by a compound, comprising:
reacting a compound of formula I-IV (“probe”) with a polypeptide, thereby alkylating the polypeptide at cysteine residues therein;
digesting the alkylated polypeptide with at least one proteolytic enzyme, thereby producing probe-labeled peptide fragments of the alkylated polypeptide;
isolating the probe-labeled peptide fragments on a solid phase support;
contacting the thus-isolated probe-labeled peptide fragments with a diboron reagent, thereby releasing/eluting probe-labeled peptide fragments at cysteine residues thereof, and
identifying the cysteine residues on the polypeptide.

In some embodiments, the solid phase support comprises magnetic beads. In some embodiments, the magnetic beads are Halo-Tag-coated and R₄ is chloroalkane. In some embodiments, the magnetic beads are streptavidin-coated and R₄ is biotin or a biotin derivative.

In some embodiments, the diboron reagent is tetrahydroxydiboron (THDB).

In some embodiments, the identifying of the cysteine residues on the polypeptide is conducted by liquid chromatography tandem mass spectrometry (LC-MS/MS).

In some embodiments, the identifying of the cysteine residues on the polypeptide is conducted by as capillary electrophoresis (CE-MS/MS).

In some embodiments, the identifying of the cysteine residues on the polypeptide is conducted by matrix-assisted laser desorption/ionization (MALID)-MS/MS.

In some embodiments, the identifying of the cysteine residues on the polypeptide is conducted by direct sample infusion-MS/MS.

In some embodiments, the polypeptide is a single protein or mixture of 2-20 proteins.

In some embodiments, the polypeptide is a polypeptide digest of a cell. In some embodiments, the cell is a human cell.

In some aspects, the present invention is directed to methods of quantifying the number of cysteine residues on a polypeptide that are targeted by a compound, comprising:
(i) reacting a compound of formula I-IV (“probe”) at a fixed concentration with a first mixture comprising one or more polypeptides to form a second mixture comprising one or more compounds of formula I-IV-polypeptide conjugates, wherein each of the compounds of formula I-IV-polypeptide conjugates comprise one or more thioether bonds;
(ii) repeating (i) for a first labelled compound of formula I-IV containing one stable isotope to form a third mixture comprising one or more isotopically labeled compounds of formula I-IV-polypeptide conjugates, wherein each of the isotopically labeled compounds of formula I-IV-polypeptide conjugates comprise one or more thioether bonds;
   a) repeating (ii) for a second labelled compound of formula I-IV containing two stable isotopes to form a fourth mixture comprising one or more isotopically labeled compounds of formula I-IV-polypeptide conjugates, wherein each of the isotopically labeled compounds of formula I-IV-polypeptide conjugates comprise one or more thioether bonds;
   b) repeating (ii) up to 6 more times for each successive compound of formula I-IV containing more than two stable isotopes;
(iii) combining the individual mixtures formed in (i) and (ii) to form a combined mixture;
(iv) enzymatically digesting the combined mixture to form a mixture of peptides comprising a combination of (a) one or more compounds of formula I-IV-polypeptide conjugates and (b) one or more isotopically labeled compounds of formula I-IV-polypeptide conjugates, whereby each conjugate is formed through one or more thioether bonds;
(v) capturing the polypeptide conjugates on a solid phase support;
(vi) contacting the thus-isolated polypeptide conjugates with a diboron reagent, thereby releasing the polypeptides;
(vii) analyzing the polypeptides via a targeted mass spectrometry assay;
(viii) detecting one or more thiolated ions, or derivatives ions thereof produced in the targeted mass spectrometry assay; and
(ix) determining target engagement stoichiometry for the compound of formula I-IV-polypeptide conjugate based on the ratio of thiolated ions, or derivative ions thereof, derived from the isotopically labeled to the unlabeled compounds of formula I-IV-peptide conjugates produced in the targeted mass spectrometry assay.

In some embodiments, the targeted mass spectrometry assay is LC-MS/MS, CE-MS/MS, MALID-MS/MS, or direct sample infusion-MS/MS.

In some embodiments, the compound of formula I-IV contains three stable isotopes. In some embodiments, the compound of formula I-IV contains four stable isotopes. In some embodiments, the compound of formula I-IV contains five stable isotopes. In some embodiments, the compound of formula I-IV contains six stable isotopes. In some embodiments, the compound of formula I-IV contains seven stable isotopes. In some embodiments, the compound of formula I-IV contains eight stable isotopes.

In some aspects, the present invention is directed to methods of quantifying the number of cysteine residues on a polypeptide that are targeted by a compound, comprising:
(i) reacting a compound of formula I-IV (“probe”) at a fixed concentration with a first mixture comprising one or more polypeptides to form a second mixture comprising one or more compounds of formula I-IV-polypeptide conjugates, wherein each of the compounds of formula I-IV-polypeptide conjugates comprise one or more thioether bonds;
(ii) repeating (i)×3 to form a third, fourth, and fifth mixtures;
(iii) enzymatically digesting the second, third, fourth, and fifth mixtures;
(iv) capturing the polypeptides on a solid phase support;
(v) isotopically labeling the captured polypeptides from the third, fourth, and fifth mixtures;
(vi) combining the individually captured polypeptides to form a combined mixture of captured polypeptides;
(vi) contacting the captured polypeptides with a diboron reagent, thereby releasing the polypeptides;
(vii) analyzing the polypeptides via a targeted mass spectrometry assay;
(viii) detecting one or more thiolated ions, or derivatives ions thereof produced in the targeted mass spectrometry assay; and
(ix) determining target engagement stoichiometry for the compound of formula I-IV-polypeptide conjugate based on the ratio of thiolated ions, or derivative ions thereof, derived from the isotopically labeled to the unlabeled compounds of formula I-IV-peptide conjugates produced in the targeted mass spectrometry assay.

In some embodiments, the targeted mass spectrometry assay is LC-MS/MS, CE-MS/MS, MALID-MS/MS, or direct sample infusion-MS/MS.

Other suitable methods and materials known in the art can also be used in the present invention are described in International Patent Application No. WO/2018/098473. The disclosure of which is incorporated herein by reference in its entirety.

### Modular Approach for Chemoproteomic Tools

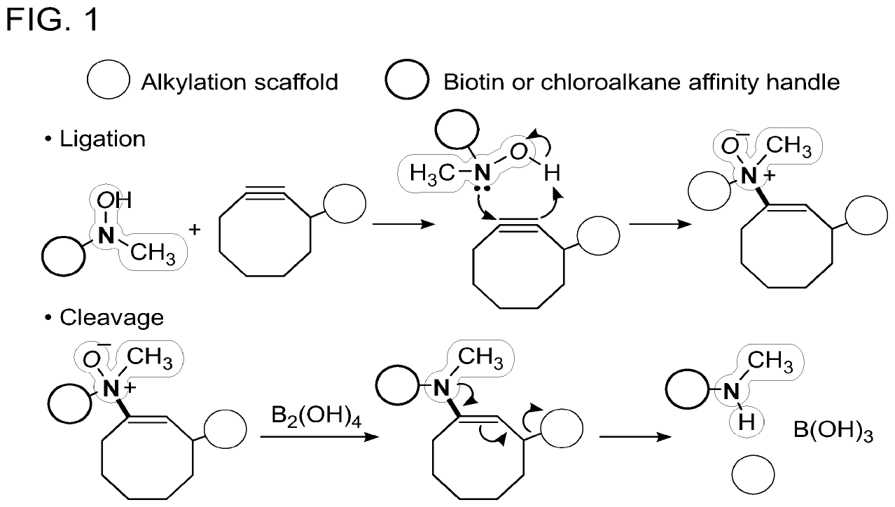

N,N-dialkylhydroxylamines and cyclooctynes have been developed as reagents for bioorthogonal ‘click-chemistry’ (Kang et al., J. Am. Chem. Soc., 143:5616-5623 (2021)) (FIG. 1) which significantly improved on current approaches. The hydroamination reaction provides second order rate constants of 84 M⁻¹s⁻¹, exquisite regioselectivity, and small reaction components. The reaction rate is ˜100× faster than strain-promoted azide-cyclooctyne reactions and comparable to the copper-catalyzed azide-alkyne variants. This scheme enables single-step, rapid and quantitative reagent ligation under mild conditions. The N,N-dialkylhydroxylamine can be reduced to as few as three non-hydrogen atoms while the cyclooctyne is likewise effective even without elaborate modification. The propargylic position is well suited for attachment of the alkylation scaffolds described herein (FIG. 1, top). An efficient bioorthogonal ligation reaction is essential to mix-and-match scaffolds and affinity handles, and to provide an unobtrusive cell-permeable reporter molecule that can modify endogenous cysteines independent of the physiochemical properties of the biochemical enrichment handle that is subsequently appended. The ligation reaction produces an enamine N-oxide that enables bioorthogonal cleavage with similarly favorable properties. The enamine N-oxides are cleaved very rapidly with diboron reagents (Kim et al., Angew. Chem. Int. Ed. Engl., 54(52):15777-15781 (2015)). The second order rate constant (1000 M⁻¹s⁻¹) is >100× faster than other bioorthogonal cleavage reactions. This facilitates the rapid, selective, and quantitative release of scaffold-modified peptides (FIG. 1, bottom). Current bioorthogonally cleavable linkers are often unstable under cellular conditions (disulfides), cleaved under harsh conditions (low pH, UV light, oxidants, etc.) that result in protein damage or nonspecific background, or require large, hydrophobic modules (azobenzene), which limits their cell permeability, interferes with reporter probe re-activity, or drives nonspecific protein binding. Also, cleavage may leave behind chemical moieties that counteract the desired physicochemical properties of the released reporters (Friedman et al., ACS Chem. Biol., 11(9):2608-2617 (2016); Rudolf et al., Curr. Opin. Chem. Biol., 17(1):110-117 (2013); Leriche et al., Bioorg. Med. Chem., 20(2):571-582 (2012)). The developed enamine N-oxides are compatible with ligated reporters carrying a wide range of functional groups. Subsequent diboron cleavage provides ‘scar-less’ release of scaffold-peptide conjugates. In this way, the modular framework provides new capabilities to systematically optimize reagents for mass spectrometry detection across different chemoproteomic workflows.

The recent successes of new covalent drugs (e.g., ibrutinib, neratinib) has reignited interest in this class of therapeutics, particularly for cancer. Development of covalent drugs relies heavily on identifying the full spectrum of on- and off-targets within the proteome, along with the specific site of inhibitor binding to each target. Currently we cannot predict the set of targetable cysteines for a given inhibitor nor the potential for covalent labeling of off-target proteins, which can lead to unintended toxicities. These limitations impede ‘target discovery’ during development of new covalent inhibitors. Similarly, once a specific protein target has been identified as being selectively bound by an inhibitor-candidate of interest, it is difficult to ascertain the fraction of target molecules in a cell or tissue that are covalently bound by the compound (‘target occupancy’). Early in the drug discovery pipeline, target occupancy data can be used to prioritize promising compounds for further development. Ultimately target occupancy data provide a quantitative link between inhibitor concentration and efficacy. Together, target discovery and occupancy data, along with toxicology and pharmacokinetic results are critical to build accurate risk profiles and ensure that only the most promising candidate drugs are advanced and ultimately nominated for clinical trials.

Mass spectrometry methods provide particularly powerful tools in covalent drug discovery. The field of chemoproteomics seeks to characterize interactions between small molecules and their protein targets. A modular approach to the design and synthesis of reagents tailored for mass spectrometry-based chemoproteomic methods to advance covalent inhibitor target discovery and target occupancy studies are described. The development of chemoproteomic assays with paired reagents accelerate prioritization and optimization of potent and selective covalent compounds earlier in the cancer drug discovery pipeline.

### Foundational Reagents for Modular Approach to Probe Design and Synthesis

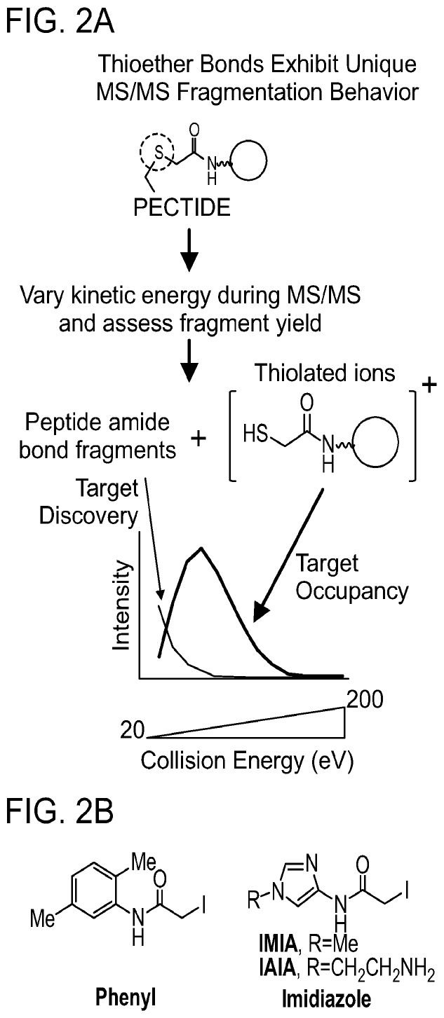

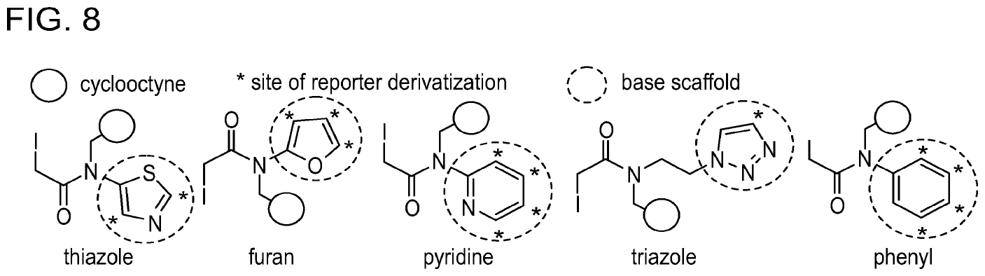

In preliminary studies a strategy for bioorthogonal ligation based on cyclooctyne hydroamination was developed. The approach provided stoichiometric ligation and subsequent cleavage with exceptionally gentle, bio-compatible reagents having a minimal chemical footprint. In parallel and building on the understanding of the unique tandem mass spectrometry (MS/MS) behavior of covalent inhibitors and clinical drugs, commercially available and newly synthesized cysteine alkylating reagents were interrogated with the objective of identifying compounds that would provide improved mass spectrometry detection and selective fragmentation, aqueous solubility, as well as a scaffold for incorporation of stable isotopes to support relative quantification studies. An imidazole scaffold, with promising physicochemical properties for target discovery and target occupancy, was integrated in the biorthogonal chemical framework to create an initial catch-and-release reagent for bio-chemical enrichment of cysteine-containing peptides. A library of alkylating scaffolds can be developed using the imidazole scaffold (FIG. 2B and Example 1) and these scaffolds are then systematically assessed as probes for target discovery via quantitative cysteine profiling with annotated covalent kinase inhibitors. Similarly, a library of heterocyclic scaffolds (FIG. 8), with a preference for structures that may impart added gas phase basicity for improved mass spectrometry detection for proteome-wide cysteine profiling, are assessed for alkylation and ionization efficiency using iodoacetamide as a control.

### Development of Multiplexed Reporter Probes to Rapidly Quantify Covalent Inhibitor Target Occupancy

The fraction of protein target molecules bound by a small molecule inhibitor provides important information to link dose and efficacy. In preliminary studies, the attributes of the cysteine alkylating reagents were leveraged to demonstrate their potential use in targeted mass spectrometry assays to quantify covalent inhibitor target-occupancy. Since the target protein and specific cysteine residue bound by the inhibitor has been established, selective fragmentation of the reporter probe scaffold was used to develop high-sensitivity, high-throughput targeted mass spectrometry assays to quantify the occupancy of the covalent inhibitor on its primary protein target. The cysteine alkylating probes are assessed as universal reagents for pulldown-based (PD) target occupancy analysis, including assay linearity and accuracy in addition to limits of detection and quantification (LOD/LOQ).

### EXAMPLES

### Example 1: Chemical Synthesis

General Information. All reactions were conducted in flame-dried round-bottom flasks under a positive pressure of nitrogen unless otherwise stated. Gas-tight syringes with stainless steel needles or cannulas were used to transfer air- and moisture-sensitive liquids. Flash column chromatography was performed using granular silica gel (60-Å pore size, 40-63 μm, Silicycle). Analytical thin layer chromatography (TLC) was performed using glass plates pre-coated with 0.25 mm silica gel impregnated with a fluorescent indicator (254 nm, Silicycle). TLC plates were visualized by exposure to short wave ultraviolet light (254 nm) and/or an aqueous solution of potassium permanganate (KMnO₄, I₂). Organic solutions were concentrated at 20° C. on rotary evaporators capable of achieving a minimum pressure of −2 torr unless otherwise stated. Room temperature is defined as 22.5 f 2.5° C. Reaction heating was performed using a UCON fluid heating bath.

Materials. All solvents were purchased from Fisher Scientific or Sigma-Aldrich. Unless otherwise stated chemical reagents were purchased from Fisher Scientific, Sigma-Aldrich, Alfa Aesar, Oakwood Chemical, Acros Organics, Combi-Blocks, or TCI America. CMA refers to a solution of 80:18:2 v/v/v chloroform:methanol:ammonium hydroxide (28-30% ammonia solution). Chloroform used in CMA solutions and as co-eluents in silica gel column chromatography were stabilized with 0.75% v/v ethanol.

General Instrumentation. Proton nuclear magnetic resonance (¹H NMR) spectra, recorded with a 500 MHz Avance III Spectrometer with multi-nuclear Smart probe, are reported in parts per million on the S scale, and are referenced from the residual protium in the NMR solvent (CDCl₃: δ 7.24, CD₃OD: δ 3.31 (CHD₂OD), CD₃CN: δ 1.94). Data are reported as follows: chemical shift [multiplicity (s=singlet, d=doublet, t=triplet, dd=doublet of doublets, dt=doublet of triplets, dq=doublet of quartets, ddd=doublet of doublets of doublets, tt=triplet of triplets, td=triplet of doublets, tq=triplet of quartets, m=multiplet), coupling constant(s) in Hertz, integration, assignment]. Carbon-13 nuclear magnetic resonance (13C NMR) spectra are referenced from the carbon resonances of the solvent (CDCl₃: δ 77.23, CD₃OD: δ 49.15, CD₃CN: δ 1.37). Fluorine-19 nuclear magnetic resonance (¹⁹F NMR) is calibrated from the fluorine resonances of benzotrifluoride (CDCl₃: δ −62.76, CD₃OD: δ −64.24). Data are reported as follows: chemical shift (assignment). Infrared data (IR) were obtained with a Cary 630 Fourier transform infrared spectrometer equipped with a diamond ATR objective and are reported as follows: frequency of absorption (cm-1), intensity of absorption (s=strong, m=medium, w=weak, br=broad).

### Cyclooctynyl Chloroacetamide

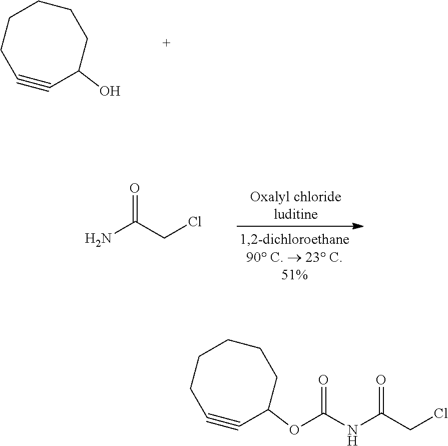 [SMILES: OC1C#CCCCCC1 ; NC(=O)CCl ; O=C(CCl)NC(=O)OC1C#CCCCCC1]

A round-bottom flask was charged sequentially with 2-chloroacetamide (56 mg, 0.60 mmol), 1,2-dichloroethane (2 mL), and oxalyl chloride (67 μL, 0.78 mmol) at 0° C. The reaction mixture was heated to 90° C. under reflux condition. After 4 h, the reaction mixture was cooled to 0° C. and lutidine (180 μL, 1.56 mmol) and cyclooctynol (74 mg, 0.60 mmol) were added. The reaction mixture was warmed to 23° C. After 12 h, the resulting reaction mixture was poured into a separatory funnel containing 1M HCl solution (10 mL) and dichloromethane (20 mL). The aqueous layer was extracted with dichloromethane (2×20 mL), and the combined organic layers were dried over anhydrous sodium sulfate, were filtered, and were concentrated under reduced pressure. The residue was purified by flash column chromatography on silica gel (eluent: 14% ethyl acetate in hexanes) to afford cyclooctynyl chloroacetamide as a white solid (75 mg, 51%) as a white. ¹H NMR (500 MHz, CD₃OD, 20° C.): δ 5.29 (m, 1H), 4.42 (s, 2H), 2.30-2.14 (m, 3H), 2.10-2.03 (m, 1H), 1.95-1.89 (m, 2H), 1.87-1.78 (m, 1H), 1.77-1.70 (m, 1H), 1.69-1.57 (m, 2H). ¹³C NMR (125.8 MHz, CD₃OD, 20° C.): δ 162.3, 152.9, 103.6, 91.2, 69.7, 44.9, 35.4, 30.8, 27.3, 21.3. FTIR (thin film) cm⁻¹: 3280 (br), 2930 (s), 2851 (w), 2217 (w), 1774 (s), 1707 (s), 1524 (br), 1449 (s), 1398 (s), 1196 (s), 1155 (s). TLC (14% ethyl acetate in hexane), Rf 0.23 (KMnO₄, I₂).

### Cyclooctynyl Iodoacetamide

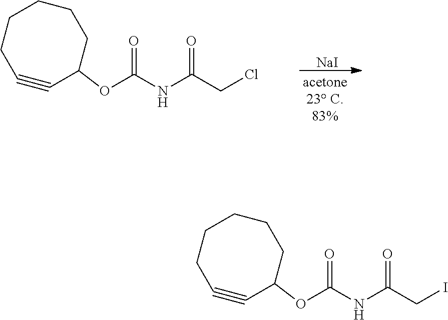 [SMILES: O=C(CCl)NC(=O)OC1C#CCCCCC1 ; "NaI" ; O=C(CI)NC(=O)OC1C#CCCCCC1]

A round-bottom flask was charged sequentially with cyclooctynyl chloroacetamide (55 mg, 0.22 mmol), acetone (4 mL), and sodium iodide (169 mg, 1.13 mmol) at 23° C. After 2.5 h, the resulting reaction mixture was concentrated under reduced pressure. The residue was purified by flash column chromatography on silica gel (eluent: 17% ethyl acetate in hexanes) to afford cyclooctynyl iodoacetamide as a yellow oil (63 mg, 83%). ¹H NMR (500 MHz, CD₃OD, 20° C.): δ 5.30 (m, 1H), 3.98 (t, J=7.7, 2H), 2.30-2.15 (m, 3H), 2.12-2.03 (m, 1H), 1.95-1.89 (m, 2H), 1.87-1.79 (m, 1H), 1.78-1.71 (m, 1H), 1.69-1.57 (m, 2H). ¹³C NMR (125.8 MHz, CD₃OD, 20° C.): δ 171.2 (C₂), 152.7 (C₁), 103.5 (C₅), 91.2 (C₆), 69.6 (C₄), 42.7 (C₁₁), 35.4 (C₈), 30.8 (C₉), 27.3 (C₁₀), 21.3 (C₇), −1.8 (C₃.). FTIR (thin film) cm⁻¹: 3276 (br), 2930 (s), 2851 (w), 1759 (s), 1696 (s), 1513 (s), 1449 (w), 1312 (w), 1200 (s), 1155 (s). TLC (14% ethyl acetate in hexane), Rf: 0.25 (KMnO₄, I₂).

In the following examples, the structures of the synthesized compounds were confirmed by ¹H NMR, ¹³C NMR, and FTIR.

### Cyclooct-2-yn-1-yl(2-aminoethyl)carbamate

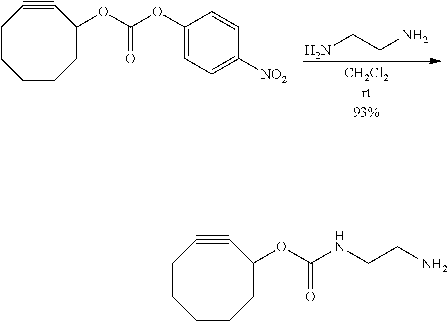

Cyclooct-2-yn-1-yl(4-nitrophenyl)carbonate (161 mg, 556 μmol, 1 equiv) was added to a solution of ethylenediamine (161 mg, 2.79 mmol, 5.00 equiv) in dichloromethane (5.50 mL) at room temperature. After 30 min, the crude mixture was diluted with 80% methanol/water (2.00 mL) and purified by automated C₁₈ reverse phase column chromatography (30 g C₁₈ silica gel, 25 μm spherical particles, eluent: gradient 0→100% MeCN/H₂O+0.1% TFA) to give cyclooct-2-yn-1-yl(2-aminoethyl)carbamate as a colorless oil (191 mg, 93%, 1:1.4 carbamate:TFA).

### Cyclooct-2-yn-1-yl(2-(2-iodoacetamido)ethyl)carbamate

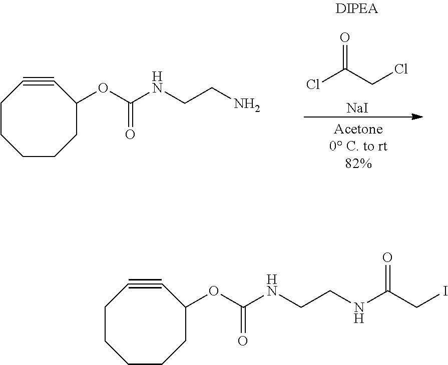

N,N-diisopropylethylamine (270 μL, 1.55 mmol, 3.00 equiv) and chloroacetyl chloride (45.2 μL, 569 μmol, 1.10 equiv) were sequentially added to a solution of cyclooct-2-yn-1-yl(2-aminoethyl)carbamate (109 mg, 517 μmol, 1 equiv) in acetone (10.0 mL) at 0° C. After 15 min, sodium iodide (384 mg, 2.56 mmol, 5.00 equiv) was added, and the solution was allowed to warm to room temperature. After 6 h, the crude mixture was diluted with hexanes (5.00 mL) and purified by flash column chromatography on silica gel (eluent: 70% ethyl acetate in hexanes) to give cyclooct-2-yn-1-yl(2-(2-iodoacetamido)ethyl)carbamate as a white solid (160 mg, 82%).

### Cyclooct-2-yn-1-yl(2-hydroxyethyl)carbamate

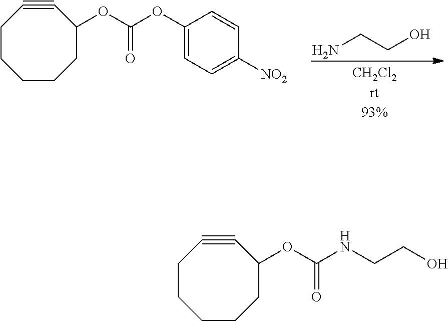

Cyclooct-2-yn-1-yl(4-nitrophenyl)carbonate (196 mg, 679 μmol, 1 equiv) was added to a solution of ethanolamine (164 μL, 3.40 mmol, 5.00 equiv) in dichloromethane (3.00 mL) at room temperature. After 30 min, the crude mixture was diluted with hexanes (1.50 mL) and purified by flash column chromatography on silica gel (eluent: 5% acetone in hexanes) to give cyclooct-2-yn-1-yl(2-hydroxyethyl)carbamate as a colorless oil (134 mg, 93%).

### Cyclooct-2-yn-1-yl(2-iodoethyl)carbamate

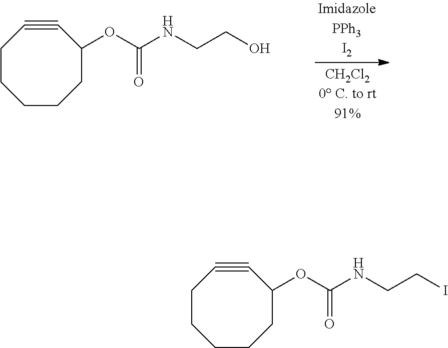 [SMILES: O=C(NCCO)OC1C#CCCCCC1 ; O=C(NCCI)OC1C#CCCCCC1]

Triphenylphosphine (173 mg, 660 μmol, 1.06 equiv) and iodine (168 mg, 660 μmol, 1.06 eq) were sequentially added to a solution of imidazole (84.8 mg, 1.25 mmol, 2.00 equiv) in dichloromethane (4 mL) at 0° C. After 30 min, a solution of cyclooct-2-yn-1-yl(2-hydroxyethyl)carbamate (132 mg, 623 μmol, 1 equiv) in dichloromethane (4.00 mL) was added via cannula. After 2 h, the crude mixture was purified by flash column chromatography on silica gel (eluent: 18% acetone in hexanes) to give cyclooct-2-yn-1-yl(2-iodoethyl)carbamate as a white solid (181 mg, 91%).

### Cyclooct-2-yn-1-yl(2-(benzylamino)ethyl)carbamate

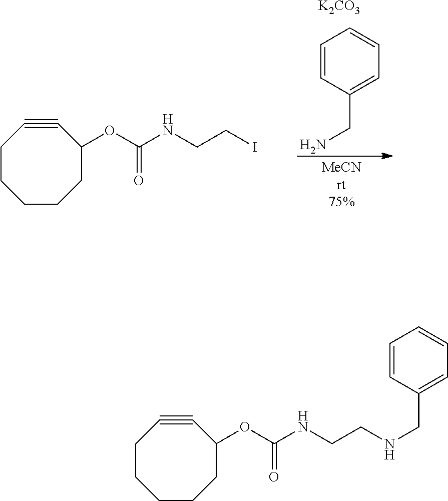 [SMILES: "K2CO3" ; O=C(NCCI)OC1C#CCCCCC1 ; NCc1ccccc1 ; O=C(NCCNCc1ccccc1)OC1C#CCCCCC1]

Cyclooct-2-yn-1-yl(2-iodoethyl)carbamate (22.0 mg, 68.5 μmol, 1 equiv) and benzylamine (29.9 uL, 274 μmol, 4.00 equiv) were sequentially added to a solution of potassium carbonate (12.3 mg, 89.1 μmol, 1.30 equiv) in acetonitrile (1.00 mL) at room temperature. After 2 days, the crude mixture was diluted with water (200 μL) and purified by automated C₁₈ reverse phase column chromatography (30 g C₁₈ silica gel, 25 μm spherical particles, eluent: gradient 0→100% MeCN/H₂O+0.1% TFA). The combined fractions were concentrated under reduced pressure, dissolved in ethyl acetate (10 mL), and washed with aqueous sodium hydroxide (1 M, 5 mL). The organic layer was dried over anhydrous sodium sulfate, filtered, and concentrated under reduced pressure to give cyclooct-2-yn-1-yl(2-(benzylamino)ethyl)carbamate as a yellow oil (15.5 mg, 75%).

### Cyclooct-2-yn-1-yl(2-(N-benzyl-2-iodoacetamido)ethyl)carbamate

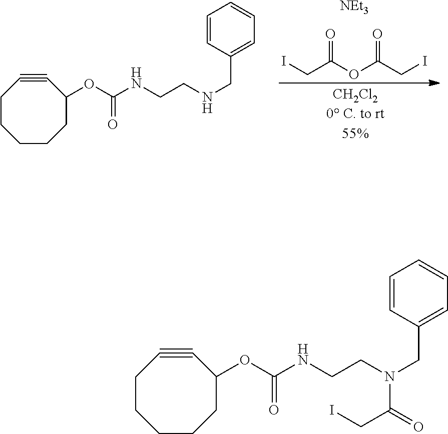

Triethylamine (13.0 μL, 92.1 μmol, 2.00 equiv) and iodoacetic anhydride (16.3 mg, 46.0 μmol, 1.00 equiv) were sequentially added to a solution of cyclooct-2-yn-1-yl(2-(benzylamino)ethyl)carbamate (15.2 mg, 50.6 μmol, 1 equiv) in dichloromethane (400 μL) at 0° C. After 2 h, the crude mixture was diluted with hexanes (1.60 mL) and purified by flash column chromatography on silica gel (eluent: 20% acetone in hexanes) to give cyclooct-2-yn-1-yl(2-(N-benzyl-2-iodoacetamido)ethyl)carbamate as a colorless oil (13.0 mg, 55%).

### Cyclooct-2-yn-1-yl(2-((furan-2-ylmethyl)amino)ethyl)carbamate

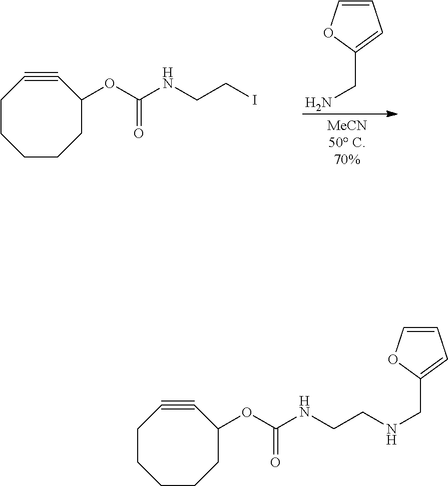 [SMILES: O=C(NCCI)OC1C#CCCCCC1 ; NCc1ccco1 ; O=C(NCCNCc1ccco1)OC1C#CCCCCC1]

Furan-2-ylmethanamine (63.5 mg, 654 μmol, 7.00 equiv) was added to a solution of cyclooct-2-yn-1-yl(2-iodoethyl)carbamate (30.0 mg, 93.5 μmol, 1 equiv) in acetonitrile (1 mL) at room temperature. The solution was then heated to 50° C. After 6 h, the crude mixture was diluted with dichloromethane (1 mL) and purified by flash column chromatography on silica gel (eluent: gradient, 2-3% methanol in dichloromethane) to give cyclooct-2-yn-1-yl(2-((furan-2-ylmethyl)amino)ethyl)carbamate as a light yellow oil (19.0 mg, 70%).

### Cyclooct-2-yn-1-yl(2-(N-(furan-2-ylmethyl)-2-iodoacetamido)ethyl)carbamate

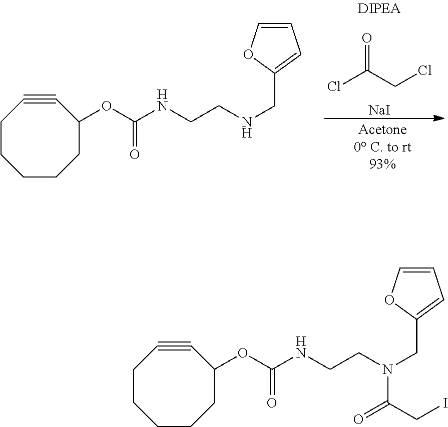

N,N-diisopropylethylamine (16.6 μL, 95.1 μmol, 1.50 equiv) and chloroacetyl chloride (5.55 μL, 70 μmol, 1.10 equiv) were sequentially added to a solution of cyclooct-2-yn-1-yl(2-((furan-2-ylmethyl)amino)ethyl)carbamate (18.4 mg, 63.4 μmol, 1 equiv) in acetone (1.00 mL) at 0° C. After 15 min, sodium iodide (47.5 mg, 317 μmol, 5.00 equiv) was added, and the solution was allowed to warm to room temperature. After 6 h, the crude mixture was diluted with hexanes (3.00 mL) and purified by flash column chromatography on silica gel (eluent: 30% acetone in hexanes) to give cyclooct-2-yn-1-yl(2-(N-(furan-2-ylmethyl)-2-iodoacetamido)ethyl)carbamate as a white solid (27 mg, 93%).

### Cyclooct-2-yn-1-yl(2-((thiophen-2-ylmethyl)amino)ethyl)carbamate

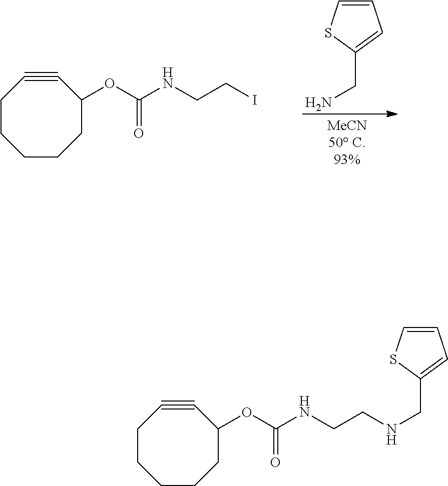 [SMILES: O=C(NCCI)OC1C#CCCCCC1 ; NCc1cccs1 ; O=C(NCCNCc1cccs1)OC1C#CCCCCC1]

Thiophene-2-ylmethanamine (73.9 mg, 654 μmol, 7.00 equiv) was added to a solution of cyclooct-2-yn-1-yl(2-iodoethyl)carbamate (30 mg, 93.5 μmol, 1 equiv) in acetonitrile (1 mL) at room temperature. The solution was then heated to 50° C. After 6 h, the crude mixture was diluted with dichloromethane (1 mL) and purified by flash column chromatography on silica gel (eluent: gradient, 2-3% methanol in dichloromethane) to give cyclooct-2-yn-1-yl(2-((thiophen-2-ylmethyl)amino)ethyl)carbamate as a light yellow oil (26.6 mg, 93%).

### Cyclooct-2-yn-1-yl(2-(2-iodo-N-(thiophen-2-ylmethyl)acetamido)ethyl)carbamate

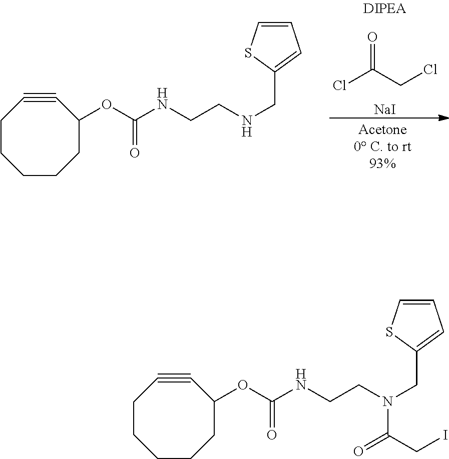

N,N-diisopropylethylamine (25.3 μL, 146 μmol, 1.50 equiv) and chloroacetylchloride (8.50 μL, 107 μmol, 1.10 equiv) were sequentially added to a solution of cyclooct-2-yn-1-yl(2-((thiophen-2-ylmethyl)amino)ethyl)carbamate (29.7 mg, 97.0 μmol, 1 equiv) in acetone (1.50 mL) at 0° C. After 15 min, sodium iodide (72.7 mg, 485 μmol, 5.00 equiv) was added, and the solution was allowed to warm to room temperature. After 6 h, the crude mixture was diluted with hexanes (5.00 mL) and purified by flash column chromatography on silica gel (eluent: 30% acetone in hexanes) to give cyclooct-2-yn-1-yl(2-(2-iodo-N-(thiophen-2-ylmethyl)acetamido)ethyl)carbamate as a white solid (37.2 mg, 81%).

### Example 2: Design and Synthesis of Probes to Accelerate the Development of Selective, Cysteine-Directed Covalent Inhibitors

### Assessment of Small Molecule Alkylating Reagents

Previous work (Ficarro et al., Anal. Chem. 88(24):12248-12254 (2016)) showed that cysteine-directed covalent inhibitors and clinical drugs followed an unusual dissociation pathway during MS/MS whereby the intact inhibitor was cleaved from the peptide backbone while carrying with it the cysteine thiol group (FIG. 2A). In addition, the yield of inhibitor derived thiolated ions maximized at MS/MS dissociation energies above those used to sequence typical tryptic peptides (FIG. 2A). The presence of thiolated ions in MS/MS spectra were used to facilitate identification of the specific cysteine residues modified by numerous covalent inhibitors (Du et al., J. Med. Chem., 63(4):1624-1641 (2020); Gao et al., Cell Chem. Biol., 25(2):135-142 (2018); Hatcher et al., Cell Chem. Biol., 25(4):460-470 (2018); Kwiatkowski et al., Nature, 511(7511):616-620 (2014); Lim et al., Angew. Chem. Int. Ed. Engl. 53(1):199-204 (2014); Lim et al., Bioorg. Med. Chem. Lett., 25(16):3382-3389 (2015); Tan et al., J. Med. Chem., 58(16):6589-6606 (2015); Xie et al., Nat. Chem. Biol., 10(12):1006-1012 (2014); Zhang et al., Chem. Biol., 19(1):140-154 (2012); Zhang et al., Nat. Chem. Biol., 12(10):876-884 (2016)). These results support the notion that generation of thiolated ions during MS/MS is common for covalent probes which modify their targets through thioether bonds. The synthesis of general use reporter probes would mimic the dissociation behavior for covalent inhibitor target discovery and target occupancy studies. Initially, a commercially available iodoacetamide phenyl compound (FIG. 2B) was shown to efficiently alkylated cysteine-containing synthetic peptides and generated robust thiolated ions but exhibited poor aqueous solubility. Next, an iodoacetamide imidazole scaffold was synthesized (FIG. 2B) and found that this reagent provided favorable properties, including: (i) potent alkylation; (ii) water solubility; (iii) selective cleavage and generation of thiolated ions at high MS/MS dissociation energy and (iv) facile incorporation of stable isotope labels for use in quantitative targeted mass spectrometry assays.

### Reporter Probe Based on Bidirectional Biorthogonal Cyclooctyne Hydroamination Chemistry

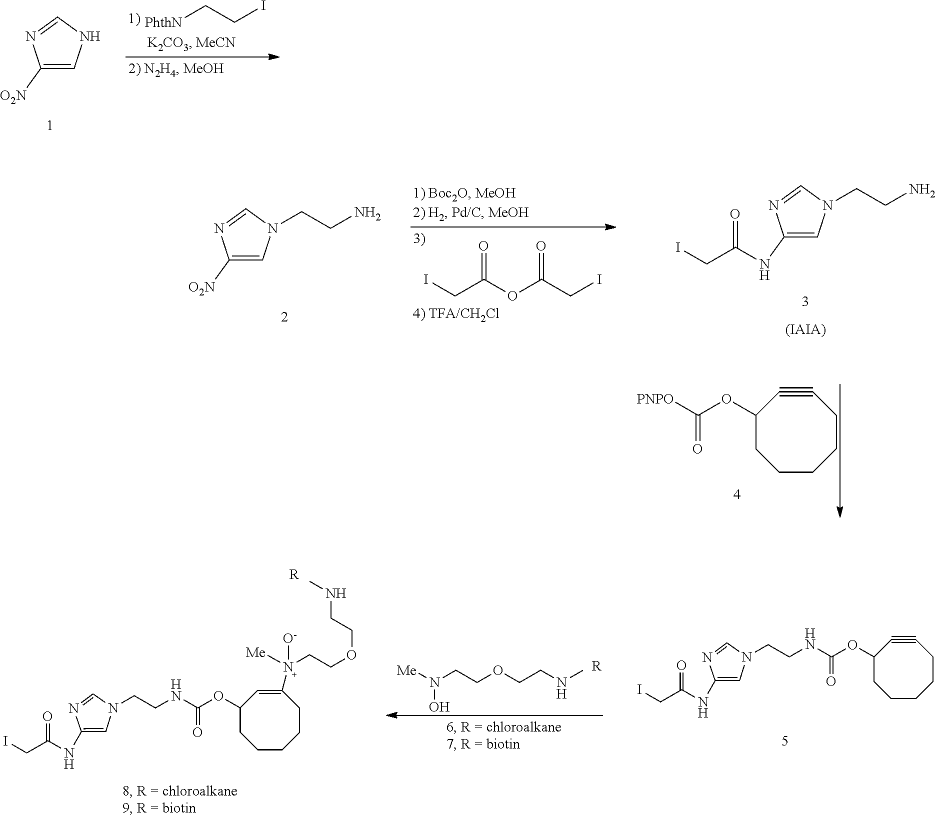

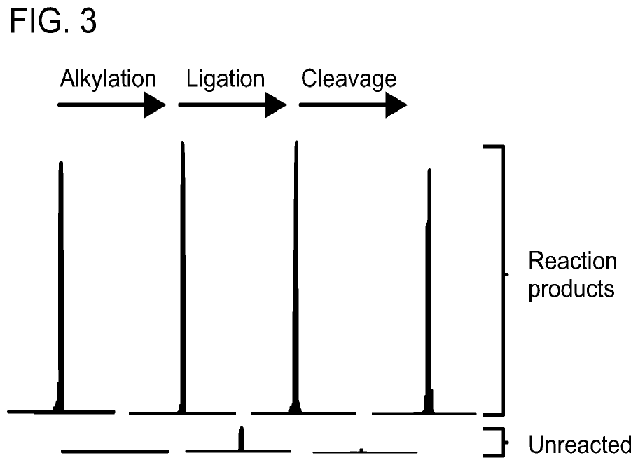

The first-generation bioorthogonal probes 8, 9 were synthesized based on the imidazole scaffold (FIG. 2B) elaborated with ethylamine. These syntheses illustrate the versatility of the bioorthogonal tools. Cyclooctyne 5, which can be used as a standalone cell-permeable alkylation reagent, when combined with biotin or HaloTag hydroxylamines 6 or 7 efficiently produced scaffold-affinity handle adducts 8 and 9, respectively. A cysteine-containing synthetic peptide was labeled with 9 and then incubated with tetrahydroxydiboron (THDB) which rapidly and quantitatively produced the aminoethyl imidazole (AIA) peptide conjugate in a scarless and MS-compatible form. MS/MS was performed across a range of kinetic energies to confirm favorable formation of thiolated fragment ions at higher kinetic energy (similar to FIG. 2A). The peptide labeling was repeated, this time beginning with the AIA-cyclooctyne 5 and continuing to the released AIA-peptide conjugate. Mass spectrometry analysis of each step (FIG. 3) demonstrated nearly quantitative efficiency for thiol alkylation, cyclooctyne ligation and cleavage, all with no detectable by-products.

### Imidazole Scaffold Used as a Catch-and-Release Reagent for Target-Discovery

As a prelude to quantitative cysteine profiling, protein extracts from human K562 cells were alkylated with compound 8 (R=chloroalkane), digested alkylated proteins with trypsin, and used Halo-Tag-coated magnetic beads to capture probe-labeled peptides. The beads were incubated with THDB to elute AIA-labeled cysteine peptides followed by LC-MS/MS analysis. Over 18,000 unique labeled cysteine sites were identified on ˜6100 proteins with a selectivity (cys-peptide/all peptides) >99%, demonstrating the suitability of the probe for cysteine profiling experiments.

### Imidazole Scaffold Used in a First-Generation Target Occupancy Probe

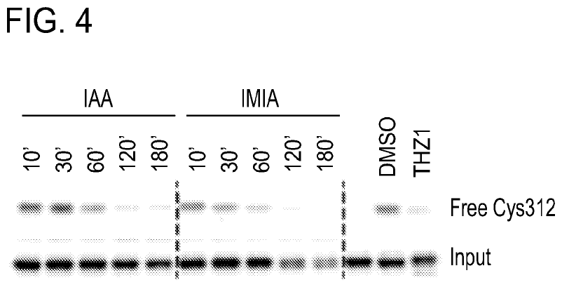

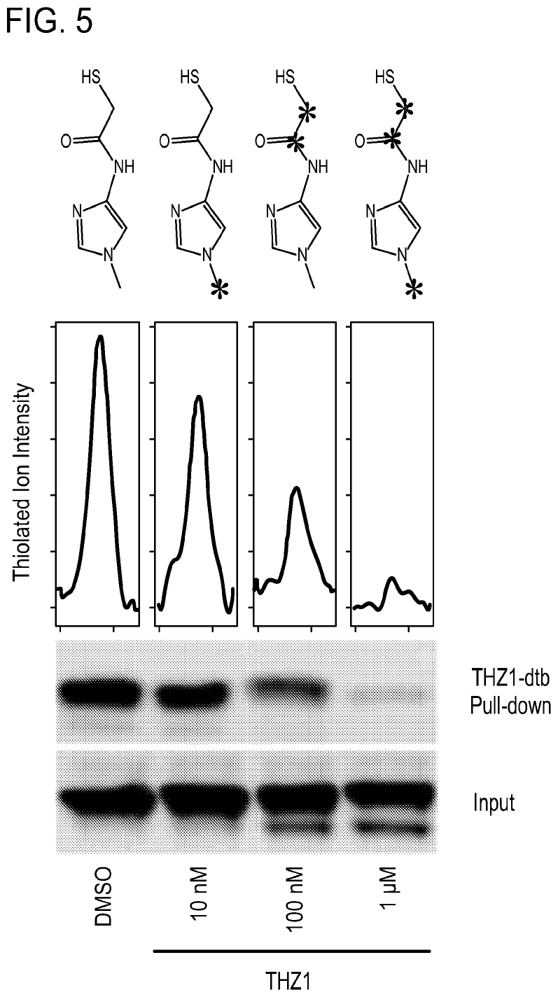

To explore whether the generation of thiolated ions during MS/MS (FIG. 2A) could be leveraged for selective detection, targeted mass spectrometry assays were employed. Initially, the methyl-imidazole iodoacetamide scaffold (‘IMIA’, FIG. 2B) was used in conjunction with antibody-based target protein enrichment. First, a cysteine synthetic peptide was used to confirm the kinetic energy-dependent generation of thiolated reporter ions (similar to FIG. 2A). Live K562 cells were treated with vehicle (DMSO), iodoacetamide (IAA, 1 mM), IMIA (1 mM), or the covalent CDK7 inhibitor (targeting cys-312), THZ1 (1 μM) (Kwiatkowski et al., Nature, 511(7511):616-620 (2014)). Protein extracts were generated and a desthiobiotin-tagged analog of THZ1 (THZ1-DTB) was used to pulldown CDK7 (FIG. 4). These data demonstrated that IMIA is a soluble, potent cysteine alkylation agent suitable for use as a probe in live cells. Next, stable isotopes were incorporated in the synthesis of IMIA, which creating a 4-channel reagent for development of a multiplexed target occupancy assay. The 4-plex IMIA reagents were used to quantify THZ1 occupancy on CDK7 (FIG. 5). Collectively these data validated selective mass spectrometry detection of thiolated ions to develop target occupancy (TO) assays.

### Example 3: Synthesis and Assay Development

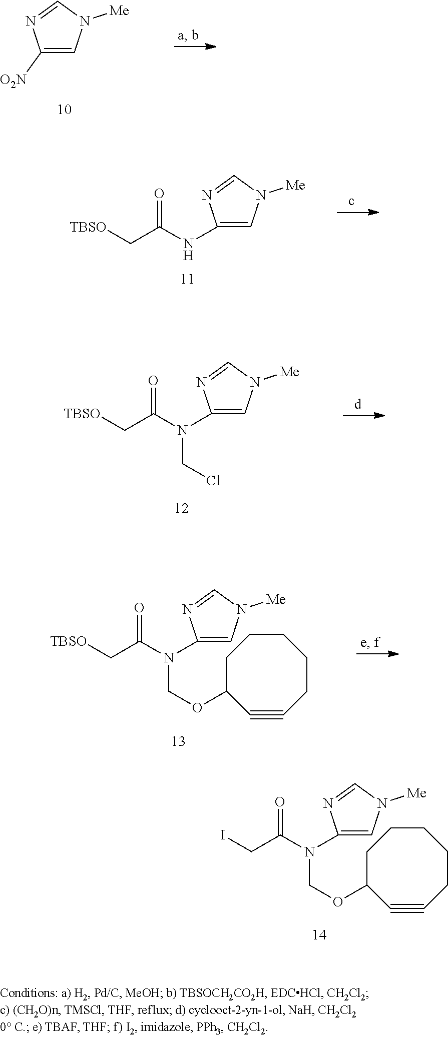 [SMILES: Cn1cnc([N+](=O)[O-])c1 ; Cn1cnc(NC(=O)CO[Si](C)(C)C(C)(C)C)c1 ; Cn1cnc(N(CCl)C(=O)CO[Si](C)(C)C(C)(C)C)c1 ; Cn1cnc(N(COC2C#CCCCCC2)C(=O)CO[Si](C)(C)C(C)(C)C)c1 ; Cn1cnc(N(COC2C#CCCCCC2)C(=O)CI)c1]

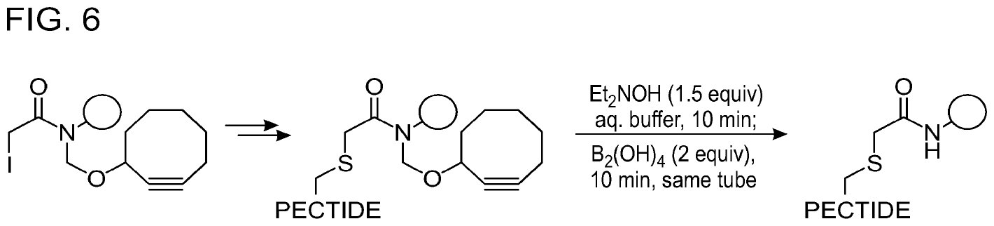

Hydrogenation of 1-methyl-4-nitroimidazole, EDC-mediated amide formation with TBS-protected glycolic acid, and chloro-methylenation with formaldehyde and chlorotrimethylsilane provides chloromethyl amide 12 (Moreira et al., Tetrahedron Lett., 35(38):7107-7110 (1994)). Deprotonation of cyclooct-2-yn-1-ol with sodium hydride and nucleophilic displacement of the chloride provides ether 13 (Smith et al., J. Org. Chem., 59(7):1719-1725 (1994)), which is then deprotected and converted to iodoacetamide 14 under Appel reaction conditions (Trost et al., J. Am. Chem. Soc., 129(47):14556-14557 (2007)). With this design, the properties of the methyl-imidazole scaffold post-capture and release remain unaltered. This approach represents a general method of derivatizing any scaffold as it is agnostic to the scaffold structure insofar as it contains a single point of attachment. Scaffolds are generated as an iodoacetamide caged with a cyclooctyne (FIG. 6). This strategy has several benefits, (i) flexibility to explore scaffold chemical space, protected from undesired reactivity with iodoacetamide; (ii) facile deprotection of cyclooctyne in one pot reaction with N,N-diethylhydroxylamine (DEHA) and tetrahydroxydiboron (THDB) that facilitates rapid evaluation of different scaffolds and chemical elaborations on standard peptides/proteins; (iii) flexibility in workflow design, live cell protein labeling followed by lysis and ligation of affinity handle, or one-step labeling and enrichment at the peptide level. As demonstrated, biotin or chloroalkane were appended for biochemical pulldown on streptavidin- or to halotag-coated beads.

For the application of inhibitor target discovery through cysteine profiling, a series of assays are used to systematically assess each scaffold for ionization efficiency, impact on peptide sequence assignment, and compatibility with widely used TMT multiplexed reagents for relative quantification. First, commercially available, isotopically encoded iodoacetamide (¹³C, ¹⁵N, ¹⁸O, heavy-IAA, Sigma, cat #638536) and cyclooctyne IAA (CO-IAA, no isotope or ‘light’) is used to alkylate separate aliquots of a cysteine-containing synthetic peptide. As in FIG. 3, the peptides are analyzed by capillary electrophoresis (CE)-MS at each step to ensure quantitative alkylation (both reagents), conjugation to DEHA and THDB cleavage (for CO-IAA). Comparison of the signals for the heavy and light carbamidomethylated peptides provide a direct assessment of the labeling and cleavage efficiencies. This assessment is repeated for the synthetic peptide labeled with each scaffold (FIG. 8). Comparison of unlabeled, input peptide with the cleaved, labeled peptide provide alkylation, ligation, and cleavage efficiency and confirm that the bidirectional bioorthogonal reagents are agnostic with respect to the scaffold structure. Next, mass spectral signal intensity (MS1) is compared from direct infusion of the synthetic peptide labeled with heavy-IAA (Sigma encoded reagent), CO-IAA, and each of the CO-scaffolds (following DEHA-ligation and THDB cleavage). This analysis establishes the potential benefit of each scaffold on peptide ionization compared to IAA as the most widely used alkylating agent in proteomic workflows. CO-IAA along with the CO-scaffolds are used to label a cysteine-containing synthetic peptide. The alkylated peptide is incubated with DEHA and then split into two equal aliquots. One aliquot is labeled with TMT0 stable isotope reagent and then cleave both aliquots independently to yield scaffold-labeled peptides in one aliquot, and TMT0/scaffold di-labeled peptides in the second aliquot. CE-MS/MS data is then acquired to verify TMT labeling and confirm the integrity of the scaffolds. This analysis provides a detailed assessment of the impact of each scaffold on peptide ionization and fragmentation and also confirm that the scaffolds are compatible with widely used stable isotope labels for relative quantification studies.

### Assess Performance of Optimized Reagents for Quantitative Cysteine Profiling Target Discovery

Base scaffolds (FIG. 8) are assessed for use in quantitative cysteine profiling for a set of covalent inhibitors that target cyclin dependent kinases (CDKs). Individual HeLa cell cultures are treated for 6 hrs with: THZ1 (CDK7 and CDK12/13) (Kwiatkowski et al., Nature, 511(7511):616-620 (2014)), YKL-05-124 (CDK7) (Olson et al., Cell Chem. Biol., 26(6):792-803 (2019)), THZ531 (CDK12/13) (Zhang et al., Nat. Chem. Biol., 12(10):876-884 (2016)), E9 (CDK12) (Gao et al., Cell Chem. Biol., 25(2):135-142 (2018)), each at 2× and 10× their respective IC₅₀, using DMSO as negative control (×2). Cells are incubated with one of the three CO-scaffolds, cell extracts are prepared, digested overnight with trypsin, peptides are de-salted and each condition are encoded with TMT10-plex isotope reagents. TMT-labeled peptides are combined, incubated with hydroxylamine-biotin (5 mM, 2 hr), probe-conjugated peptides are captured on magnetic streptavidin beads (1 mL, 50 nmol capacity), peptides are washed and eluted using THDB (20 mM, 30 min). Peptides are analyzed by multidimensional reversed phase (RP)-strong anion exchange (SAX)—RP LC-MS/MS on a Q Exactive HF (Kim et al., Elife. 9 (2020); Brown et al., Cancer Discov., 8(4):478-497 (2018); Norberg et al., Cell Death Differ., 24(2):251-262 (2017); Hsu et al., Cancer Res., 77(17):4613-4625 (2017); Wang et al., Blood, 128(11):1465-1474 (2016); Lu et al., Cell Stem Cell, 15(1):92-101 (2014); Zhou et al., Nat. Commun., 4:2171 (2013); Lee et al., EMBO J., 31(10):2403-2415 (2012); Zhou et al., Anal. Chem., 83(18):6996-7005 (2011); Hsu et al., Science, 332(6035):1317-1322 (2011); Ficarro et al., Mol. Cell. Proteomics, 10(11):O111 011064 (2011)). This experiment is repeated for the other two CO-scaffolds. The MS/MS data is searched using Mascot to identify peptides (<1% FDR). The total number of unique cysteine sites profiled are compared as well as the distribution of peptide sequence scores in each dataset. Extracted ion chromatograms are generated for all high-confidence peptides taking care to annotate the contribution of each charge state to the total peptide signal. The differences in sequence composition, length, isoelectric point and predicted hydrophobicity for peptides labeled with each CO-scaffold are explored. Finally, cysteine binding is correlated for each inhibitor (statistics assigned based on variance model derived from two DMSO control channels (Zhang et al., Mol. Cell. Proteomics, 9(5):780-790 (2010))) with published data reporting their respective on- and off-targets (Gao et al., Cell Chem. Biol., 25(2):135-142 (2018); Kwiatkowski et al., Nature, 511(7511):616-620 (2014); Zhang et al., Nat. Chem. Biol., 12(10):876-884 (2016); Olson et al., Cell. Chem. Biol., 26(6):792-803 (2019)). This analysis assesses, across a large set of cysteine peptides, the impact of each scaffold on peptide sequence identification, peptide ionization, and also reveals potential preference of each scaffold for physiochemically distinct classes of cysteine peptides.

### Example 4: Alternate Route for Functionalizing Reporter Scaffolds and Alternative Assays

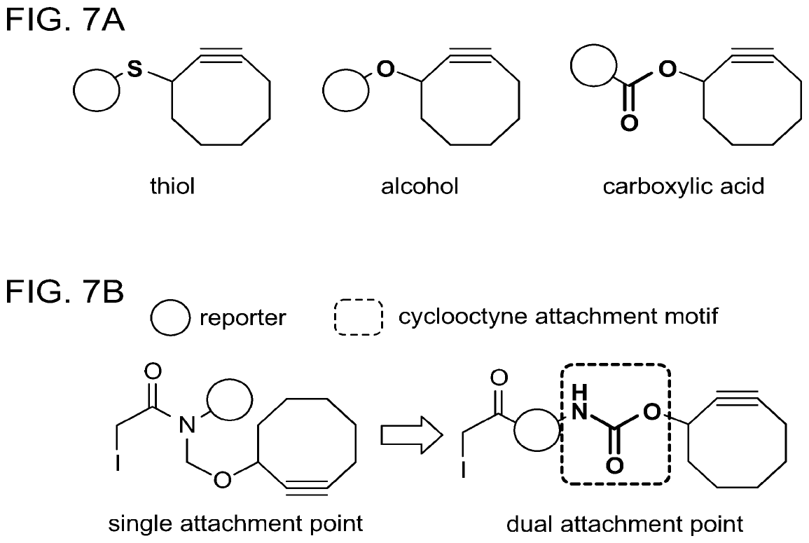

The scaffold can be attached via the iodoacetamide nitrogen; the scaffold is linked to cyclooctyne through an alternative moiety (FIG. 7). Some scaffolds generate predominantly high charge state peptides that provide poor sequence scores, lead to degenerate complexity in MS1 spectra, or result in ‘dilution’ of peptide signal across multiple charge states. Electron-transfer dissociation (ETD) during MS/MS or other scaffolds (e.g., furan) that lack ionizable nitrogens are employed as alternative strategies. For compression of thiolated ion/iTRAQ reporters, 3D fractionation depth can be increased or MS3 on the Orbitrap Lumos™ is used.

### Example 5: Development of Multiplexed Reporter Probes to Rapidly Quantify Covalent Inhibitor Target-Occupancy

Affinity-tagged inhibitor analogs are used in combination with the native inhibitor (no affinity handle) to assess inhibitor target occupancy (TO) (Barf et al., J. Pharmacol. Exp. Ther., 363(2):240-252 (2017); Evans et al., J. Pharmacol. Exp. Ther., 346(2):219-228 (2013)). The tagged/native inhibitor combination are used in different assay formats (ELISA, FRET, etc.). However, each inhibitor requires its own paired affinity-tagged analog. Developing these analogs is resource intensive and often requires detailed structure activity relationship data. As a result, this approach is usually reserved for late-stage compounds.

The general use probes described herein facilitate TO analysis at any stage in the drug discovery pipeline. The selectivity and high yield of thiolated ‘reporter’ ions generated at high collision energy during MS/MS are used to develop probes for a TO assay that is inhibitor and target agnostic. The energy-dependent yield of thiolated reporters is dependent on the base scaffold (imidazole, pyridyl, etc.) and its substituents (—CH3, —NH2, etc.). The modular synthesis scheme is used to rapidly elaborate scaffolds and optimize reporter performance. Stable isotopes are incorporated directly in the reagents to support multiplexed TO assays and circumvent commercial isobaric tag reagents. Reagents are used in combination with covalent inhibitors of DUBs to characterize the new TO assay.

### Chemically Elaborate Alkylation Scaffolds and Assess Yield of Thiolated Reporter Ions

The base scaffolds (FIG. 8) are substituted with electron donating (—OH, —OR, —CH₃), withdrawing (—CN, —COOH, —NO₂), or ionizable (—NH₂) groups to further improve and fine tune the yield of thiolated reporter ions. Each modified scaffold is synthesized as the caged cyclooctyne (FIG. 6) to avoid undesired reactivity between the iodoacetyl group and scaffold moieties (e.g., primary amine). Representative experiments are described for the iodo-acetamide imidazole scaffold elaborated with methyl- and aminoethyl-moieties (IMIA and IAIA, FIG. 2B). The synthetic cysteine peptide is alkylated with CO-IMIA and CO-IAIA and the cyclooctyne is converted to the enamine N-oxide using diethylhydroxylamine (DEHA). After tetrahydroxide diboron (THDB) release of the MIA- and AIA-labeled peptides, targeted LC-MS/MS is performed whereby the acquisition method cycles through MS1 scans followed by sequential low-/high-energy MS/MS for each labeled peptide, respectively. The low energy MS/MS scan provides peptide sequence (necessary for MS3 experiments below), with parameters for the high energy MS/MS scan based initially on the preliminary data (similar to FIG. 2A) and adjusted as necessary for other scaffolds to maximize the thiolated ion signal. K562 cells are treated with CO-IMIA and CO-IAIA and monitor CDK7 cys-312 alkylation as in FIG. 4. These experiments are repeated for each scaffold. For TO assay development (see below) the scaffold that provided the best combination of thiolated ion yield and aqueous solubility for live cell alkylation was used. Finally, stable isotopes (¹³C, ¹⁵N, ¹⁸O) were incorporated into the scaffold using commercially available building blocks to create a multiplexed reagent for the TO assay (similar to FIG. 5).

### Optimized Scaffold to Develop PD-Based Target Occupancy Assay

Target occupancy is compared for a covalent DUB inhibitor as measured by (strategy #1) isotopically encoded thiolated ions from the new reporters and (strategy #2) commercial iTRAQ 4Plex reagents, with (strategy #3) the combination of native-/affinity tagged-inhibitor and western blot as a reference for TO. Four independent cultures (prepared in triplicate, for strategy #1, #2, and #3) of MM1.S cells are treated for 6 hrs with XL177A (Schauer et al., Sci. Rep., 10(1):5324 (2020)), a selective covalent inhibitor (IC₅₀=0.3 nM) of the DUB, USP7 (1 nM, 10 nM and 100 nM) with DMSO as a negative control. After inhibitor wash-out each of the 4 treated cultures in the strategy #1 samples are incubated with one ‘channel’ of the 4Plex CO-scaffold reagent (1 mM, 1 hr). For strategy #2 samples, all of the 4 treated cultures are incubated with the ‘light’ version of the CO-scaffold reagent (1 mM, 1 hr). For strategy #3 samples, individual cell extracts are prepared after inhibitor treatment and analyze target occupancy using desthiobiotin-tagged XL177A and western blot (Schauer et al., Sci. Rep., 10(1):5324 (2020)). For strategy #1, extracts are prepared from combined cells, incubated with hydroxylamine-biotin (5 mM, 2 hrs) and proteins are digested with trypsin. For strategy #2, cell extracts are prepared separately for each treatment condition, incubated with hydroxylamine-biotin, proteins are digested with trypsin, the resulting peptides are encoded with iTRAQ 4Plex stable isotope reagents, and the peptides are combined. Desalted probe-labeled peptides are captured on streptavidin-coated beads (1 mL, 50 nmol capacity) separately for samples from strategy #1 (treatment encoded by isotopes in scaffold) and strategy #2 (treatment encoded by iTRAQ reagents). After washing beads, cysteine peptides (THDB, 20 mM, 30 min) are released. Peptides for strategy #1 and strategy #2 are analyzed using targeted MS/MS and MS3 on the Orbitrap Lumos™ instrument. For MS3 acquisition, peptide fragment ions detected in MS/MS which span the scaffold-labeled cysteine and either perform high energy fragmentation in the MS3 stage to drive production of isotopically encoded thiolated ions (strategy #1) or ‘standard’ energy for scaffold-/iTRAQ-di-labeled peptides (strategy #2) are selected. Targeted MS/MS data is also acquired on the Bruker timsTOF Pro instrument for peptides from strategy #1 and #2. The integrated trapped ion mobility separation stage on this instrument has been reported to provide accurate ratios for TMT 6Plex reporters (e.g., reporters separated by 1 Da) for MS/MS spectra (Ogata et al., Anal. Chem., 92(12):8037-8040 (2020)). Finally, to explore parameters for higher throughput, the TO assay will be repeated across LC gradients of 90, 60, 30, and 15 minutes. The accuracy of ratios, reporter signal to noise ratio (thiolated ions or iTRAQ) for each LC gradient length, are evaluated on each instrument and for each labeling strategy. TO measured using strategy #3 serves as a positive control.

### Accuracy, Linearity, and Limit of Detection/Quantification (LOD/LOQ) for TO Assay

Recombinant CDK7 (Life Tech.) is incubated with THZ1 (Kwiatkowski et al., Nature, 511(7511):616-620 (2014)) to fully engage Cys-312 (FIG. 4). Fully labeled CDK7 is combined with untreated CDK7 to achieve target occupancies of 100% (fully labeled CDK7), 90%, 10% and 0% (no labeled CDK7), keeping the total amount of CDK7 constant at 100 ng across samples. Each sample is alkylated with one channel of the 4Plex CO-scaffold reagent. The samples are combined, incubated with hydroxylamine-biotin and proteins are digested with trypsin. To provide a complex proteome background that lacks endogenous CDK7, dithiothreitol-treated E. Coli extracts (500 μg) are alkylated with one channel of the 4Plex CO-scaffold reagent. The biotin handle is appended as above, and E. coli proteins are digested with trypsin. CDK7 and E. Coli digests are combined, enriched with cysteine peptides using streptavidin-coated beads and peptides are released with THDB. LC-MS/MS methods are used to measure THZ1 occupancy on CDK7 across 5 independent replicates. The 4-point occupancy titration is repeated after reducing the total amount of CDK7 to 25 ng, 5 ng, and 1 ng. The data is used to evaluate accuracy, linearity and LOD/LOQ for the TO assay across 5 replicates.

### Target Discovery and Target Occupancy

The chemoproteomic assays require peptides to pass a 1% false discovery rate (FDR) for identification. In addition, statistics for peptide ratios are assigned using computational frameworks that have been published (Lee et al., EMBO J., 31(10):2403-2415 (2012); Lee et al., Mol. Cell, 54(3):512-525 (2014); Zhang et al., Mol. Cell. Proteomics, 9(5):780-790 (2010)); p-val≤0.05 and fold-change ≥2 std. dev. relative to the median ratio generally represent robust thresholds for mass spectrometry data (Hsu et al., Cancer Res., 77(17):4613-4625 (2017); Wang et al., Blood, 128(11):1465-1474 (2016); Lu et al., Cell Stem Cell, 15(1):92-101 (2014); Lee et al., EMBO J., 31(10):2403-2415 (2012); Caro et al., Cancer Cell, 22(4):547-560 (2012); Norberg et al., Cell Death Differ., 24(2):251-262 (2017); Park et al., Nat. Struct. Mol. Biol., 22(9):703-711 (2015); Zhang et al., Cell Stem Cell, 19(1):66-80 (2016)). Only peptides that map to unique gene identifiers in the SwissProt database of human proteins are considered (Askenazi et al., Proteomics, 10(9):1880-1885 (2010); Rozenblatt-Rosen et al., Nature, 487(7408):491-495 (2012)) for the purposes of quantification. The performance of the target discovery experiments is assessed at the level of total cysteines identified per experiment (compared to published mass spectrometry-based cysteine profiling studies) as well as the binding of each covalent inhibitor to its previously reported targets (e.g., THZ1: CDK7, CKD12, CDK13, PKN3, etc.). The performance of the target occupancy assay is assessed by direct comparison to an equivalent mass spectrometry experiment based on widely used iTRAQ isotope reagents, as well as a bio-chemical approach based on use of a DUB activity probe and western blot (Gao et al., Cell Chem. Biol., 25(2):135-142 (2018); Kwiatkowski et al., Nature, 511(7511):616-620 (2014); Zhang et al., Nat. Chem. Biol., 12(10):876-884 (2016); Olson et al., Cell. Chem. Biol., 26(6):792-803 (2019)). The linear dynamic range, accuracy, and LOD/LOQ are established for the target occupancy assay.

### Example 6: High-Content, Multiplexed Assay

Tissue-culture cells were treated for 6 hours with DMSO (control reference), a pan deubiquitinating enzyme inhibitor, Ibrutinib (a clinical inhibitor of the kinase Bruton's Tyrosine Kinase) and a pan-kinase inhibitor at two different concentrations and in duplicate. Protein extracts from treated cells were prepared in a triethyl bicarbonate buffer containing SDS and benzonase. Extracts from compound-treated cells were alkylated without prior reduction with one of the three cysteine-reactive chemical barcodes (COBRA reagents) and digested overnight with trypsin. Tryptic peptides derived from DMSO-treated or from each compound-treated samples were labeled with isobaric tags to encode treatment concentrations and replicates. Isobaric-labeled and COBRA-encoded peptides were combined in one pool, purified by solid phase extraction and subjected to enrichment using magnetic beads coated with hydroxylamine. Captured peptides were eluted from the beads using tetra-hydroxy diboron and analyzed by multidimensional LC-MS/MS. The mass spectrometry data was searched against a human database where the masses of the COBRA and the isobaric tags were considered as variable and fixed modifications, respectively. The isobaric reporter intensities were used to calculate the intensity of COBRA-labeled peptides in each treatment condition relative to the reference condition and derived the average dose-response across replicates. These ratios were transformed into Z-scores prior to plotting.

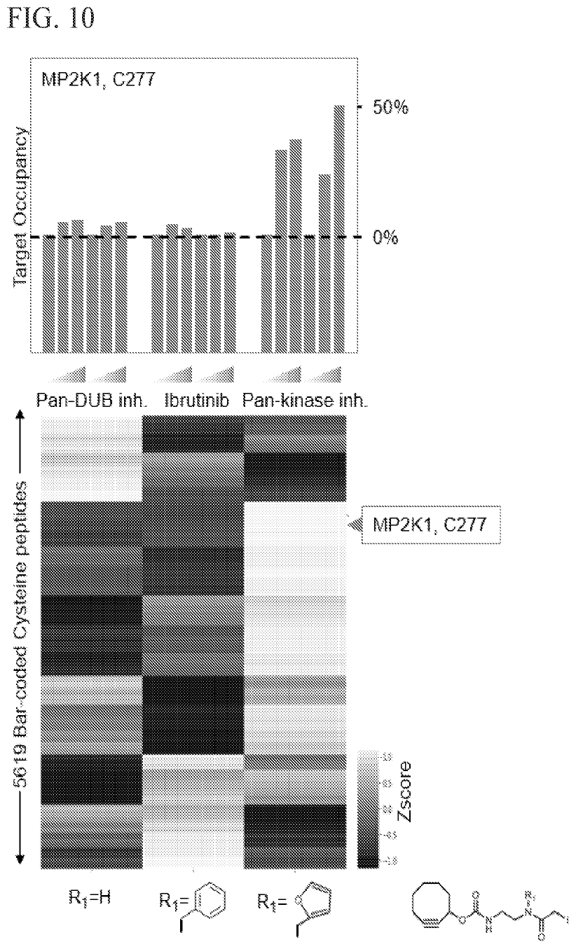

Cluster map visualization of the dose-response relationship between treatment concentration for each cysteine-targeting covalent inhibitor and site occupancy across 5916 cysteine-containing peptides is shown in FIG. 10. This map demonstrates how compounds of formulas I and II

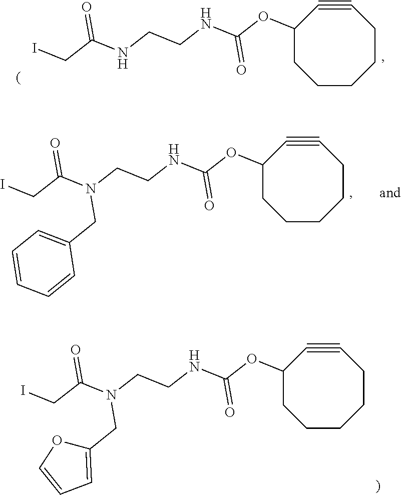 [SMILES: O=C(CI)NCCNC(=O)OC1C#CCCCCC1 ; O=C(NCCN(Cc1ccccc1)C(=O)CI)OC1C#CCCCCC1 ; O=C(NCCN(Cc1ccco1)C(=O)CI)OC1C#CCCCCC1]

can be used as cysteine-reactive barcodes in multiplexed experiments to investigate the cellular targets of multiple cysteine-reactive drugs in a single, high-content LC-MS/MS chemoproteomic assay. The modular nature of these reagents facilitates incorporation of chemical diversity (e.g., imidazolyl, thiazolyl, furanyl, pyridinyl, triazolyl, phenyl, and other heteroaryl groups) while maintaining the same warhead and bioorthogonal components. Therefore, multiple reagents can be used as ‘chemical bar codes’ to distinguish binding activity of different covalent inhibitors against the same amino acid in the same protein sequence. This approach enables development of high-content chemical proteomic screens whereby the binding behavior of multiple covalent inhibitors, probes, or fragments against the entire human proteome can be assessed in a single assay.

All patent publications and non-patent publications are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A compound which is of formula I or II: 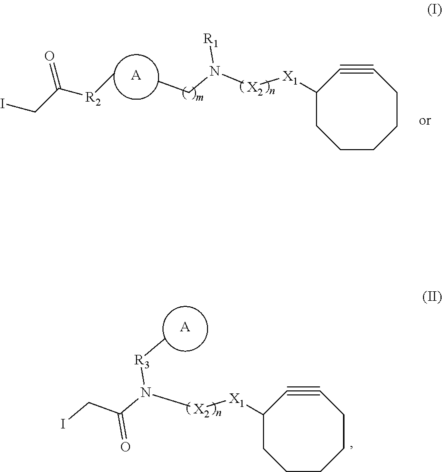
wherein,
X₁ is NR₁, O, S, S(O), or S(O)₂;
each X₂ is independently C(R₁)₂, NR₁, O, C(O), C₆-C₁₀ aryl, or —OCH₂CH₂—;
each R₁ is independently hydrogen, C₁-C₆ alkyl, C₆-C₁₀ aryl, or 5- to 10-membered heteroaryl, wherein said alkyl, aryl, or heteroaryl is optionally substituted;
R₂ is absent or NH;
R₃ is absent, C(O), or C₁-C₃ alkylene;
A is C₁-C₆ alkyl, C₆-C₁₀ aryl, 5- to 10-membered heteroaryl or a small molecule, wherein
said alkyl, aryl, or heteroaryl is optionally substituted;
m is an integer from 0-5; and
n is an integer from 0-10,
or a pharmaceutically acceptable salt or stereoisomer thereof.

2-5. (canceled)

6. The compound of claim 1, wherein R₃ is C₁-C₂ alkylene.

7-8. (canceled)

9. The compound claim 1, wherein each carbon, nitrogen, and oxygen is substituted with a stable isotope thereof, wherein the isotope is selected from ¹³C, ¹⁵N, and ¹⁸O.

10. The compound of claim 1, wherein the compound contains 1-8 isotopes.

11-12. (canceled)

13. The compound of claim 1, wherein A is imidazolyl, thiazolyl, furanyl, pyridinyl, triazolyl, or phenyl, and wherein A is optionally substituted.

14. The compound of claim 13, which is substituted with one or more electron donating groups.

15. (canceled)

16. The compound of claim 13, which is substituted with one or more electron withdrawing groups.

17. (canceled)

18. The compound of claim 13, which is substituted with one or more ionizable groups.

19. (canceled)

20. The compound of claim 1, which is

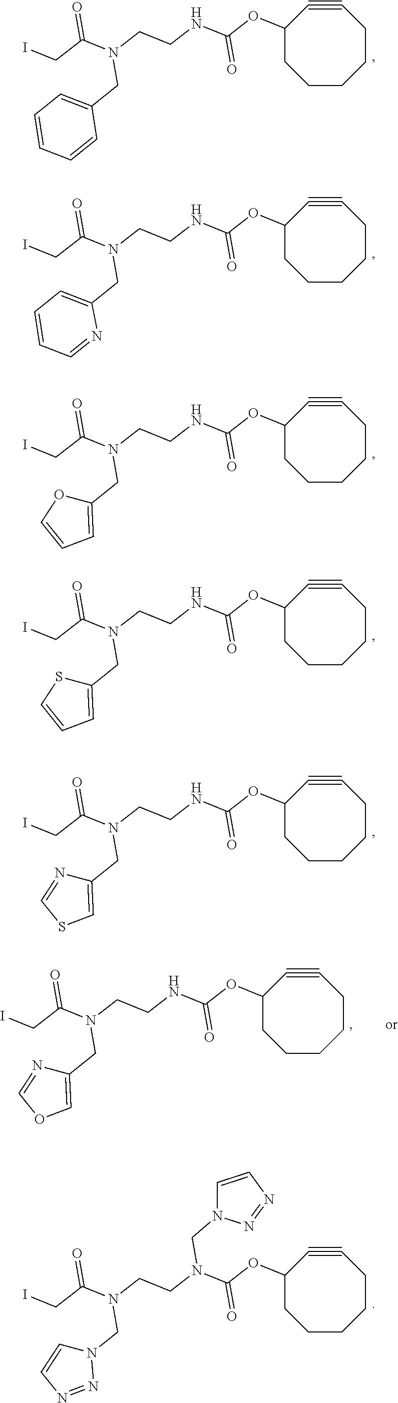

21. A composition, comprising a compound which is of formula I or II: 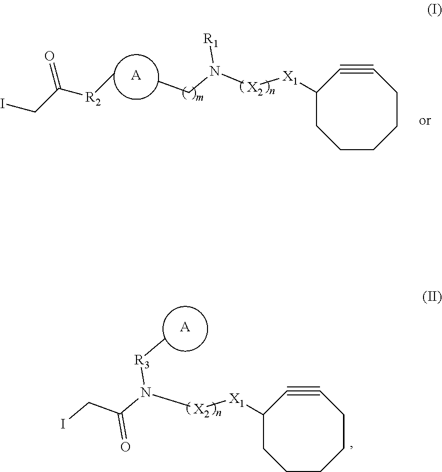
wherein:
X₁ is NR₁, O, S, S(O), or S(O)₂;
each X₂ is independently C(R₁)₂, NR₁, O, C(O), C₆-C₁₀ aryl, or —OCH₂CH₂—;
each R₁ is independently hydrogen, C₁-C₆ alkyl, C₆-C₁₀ aryl, or 5- to 10-membered heteroaryl, wherein said alkyl, aryl, or heteroaryl is optionally substituted;
R₂ is absent or NH;
R₃ is absent, C(O), or C₁-C₃ alkylene;
A is C₁-C₆ alkyl, C₆-C₁₀ aryl, 5- to 10-membered heteroaryl or a small molecule, wherein said alkyl, aryl, or heteroaryl is optionally substituted;
m is an integer from 0-5; and
n is an integer from 0-10,
or pharmaceutically acceptable salt or stereoisomer thereof and a carrier.

22. A compound which is of formula III or IV: 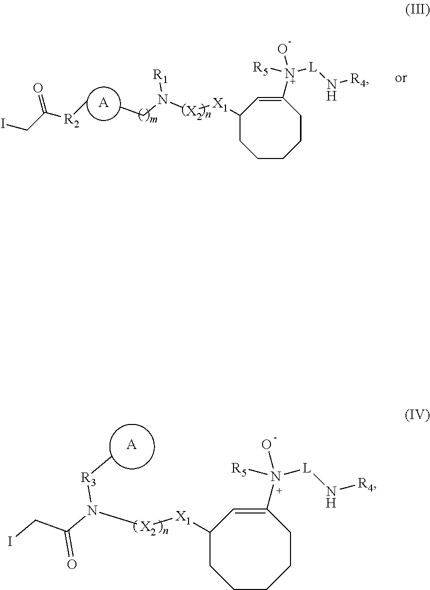
wherein,
X₁ is NR₁, O, S, S(O), or S(O)₂;
each X₂ is independently C(R₁)₂, NR₁, O, C(O), C₆-C₁₀ aryl, or —OCH₂CH₂—;
each R₁ is independently hydrogen, C₁-C₆ alkyl, C₆-C₁₀ aryl, or 5- to 10-membered heteroaryl, wherein said alkyl, aryl, or heteroaryl is optionally substituted;
R₂ is absent or NH;
R₃ is absent, C(O), or C₁-C₃ alkylene;
R₄ is an affinity handle or a bead;
R₅ is C₁-C₆ alkyl;
L is an alkylene chain or a PEG chain;
A is C₁-C₆ alkyl, C₆-C₁₀ aryl, C₆-C₁₀ arene, 5- to 10-membered heteroaryl, 5- to 10-membered heteroarylene, or a small molecule, wherein said alkyl, aryl, or heteroaryl is optionally substituted;
m is an integer from 0-5; and
n is an integer from 0-10,
or a pharmaceutically acceptable salt or stereoisomer thereof.

23-29. (canceled)

30. The compound of claim 22, wherein R₄ is:
a chloroalkane (HaloTag);
biotin or a biotin derivative;
a protein;
SNAP-tag or CLIP-tag; or
a magnetic bead, a polystyrene bead, or an agarose bead.

31-46. (canceled)

47. A process of preparing a compound of formula III: 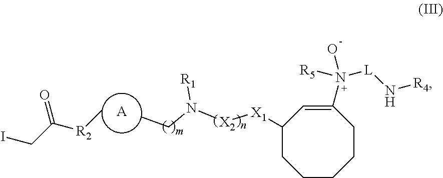
comprising reacting a compound of formula I: 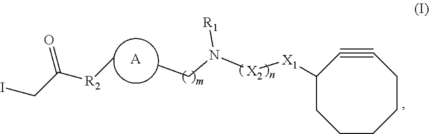
with a compound of formula V: 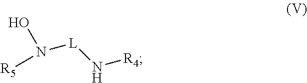
wherein,
X₁ is NR₁, O, S, S(O), or S(O)₂;
each X₂ is independently C(R₁)₂, NR₁, O, C(O), C₆-C₁₀ aryl, or —OCH₂CH₂—;
each R₁ is independently hydrogen, C₁-C₆ alkyl, C₆-C₁₀ aryl, or 5- to 10-membered heteroaryl, wherein said alkyl, aryl, or heteroaryl is optionally substituted;
R₂ is absent or NH;
R₃ is absent, C(O), or C₁-C₃ alkylene;
R₄ is an affinity handle or a bead;
R₅ is C₁-C₆ alkyl;
L is an alkylene chain or a PEG chain;
A is C₁-C₆ alkyl, C₆-C₁₀ aryl, C₆-C₁₀ arene, 5- to 10-membered heteroaryl, 5- to 10-membered heteroarylene, or a small molecule, wherein said alkyl, aryl, or heteroaryl is optionally substituted;
m is an integer from 0-5; and
n is an integer from 0-10.

48. A process of preparing a compound of formula IV: 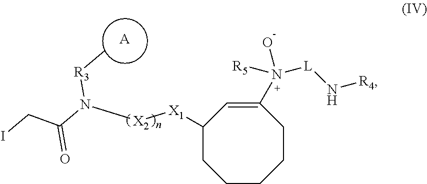
comprising reacting a compound of formula II: 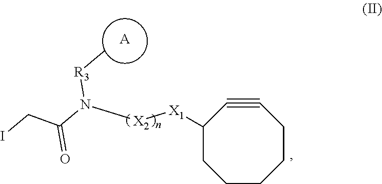
with a compound of formula V: 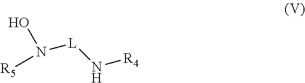
wherein,
X₁ is NR₁, O, S, S(O), or S(O);
each X₂ is independently C(R₁)₂, NR₁, O, C(O), C₆-C₁₀ aryl, or —OCH₂CH₂—;
each R₁ is independently hydrogen, C₁-C₆ alkyl, C₆-C₁₀ aryl, or 5- to 10-membered heteroaryl, wherein said alkyl, aryl, or heteroaryl is optionally substituted;
R₂ is absent or NH;
R₃ is absent, C(O), or C₁-C₃ alkylene;
R₄ is an affinity handle or a bead;
R₅ is C₁-C₆ alkyl;
L is an alkylene chain or a PEG chain;
A is C₁-C₆ alkyl, C₆-C₁₀ aryl, C₆-C₁₀ arene, 5- to 10-membered heteroaryl, 5- to 10-membered heteroarylene, or a small molecule, wherein said alkyl, aryl, or heteroaryl is optionally substituted;
m is an integer from 0-5; and
n is an integer from 0-10.

49-62. (canceled)

63. A method of identifying cysteine residues on a polypeptide that may be targeted by a compound, comprising:
reacting the compound of claim 22 with the polypeptide, thereby alkylating the polypeptide at cysteine residues therein;
digesting the alkylated polypeptide with at a proteolytic enzyme, thereby producing probe-labeled peptide fragments of the alkylated polypeptide;
isolating the probe-labeled peptide fragments on a solid phase support;
contacting the thus-isolated probe-labeled peptide fragments with a diboron reagent, preferably tetrahydroxydiboron (THDB), thereby releasing/eluting probe-labeled peptide fragments at cysteine residues thereof, and
identifying the cysteine residues on the polypeptide.

64. The method of claim 63, wherein the solid phase support comprises magnetic beads.

65. The method of claim 64, wherein i) the magnetic beads are Halo-Tag-coated and R₄ is chloroalkane or ii) the magnetic beads are streptavidin-coated and R₄ is biotin or a biotin derivative.

66-67. (canceled)

68. The method of claim 63, wherein the identifying of the cysteine residues on the polypeptide is conducted by liquid chromatography tandem mass spectrometry (LC-MS/MS), capillary electrophoresis (CE-MS/MS), matrix-assisted laser desorption/ionization (MALID)-MS/MS, or direct sample infusion-MS/MS.

69-74. (canceled)

75. A method of quantifying the number of cysteine residues on a polypeptide that are targeted by a compound, comprising:
(i) reacting the compound of claim 1 at a fixed concentration with a first mixture comprising one or more polypeptides to form a second mixture comprising one or more compounds of claim 1-polypeptide conjugates, wherein each of the compounds of claim 1-polypeptide conjugates comprise one or more thioether bonds;
(ii) repeating (i) for a first labelled compound of claim 1 containing one stable isotope to form a third mixture comprising one or more isotopically labeled compounds of claim 1-polypeptide conjugates, wherein each of the isotopically labeled compounds of claim 1-polypeptide conjugates comprise one or more thioether bonds;
a) repeating (ii) for a second labelled of claim 1 containing two stable isotopes to form a fourth mixture comprising one or more isotopically labeled compounds of claim 1-polypeptide conjugates, wherein each of the isotopically labeled compounds of claim 1-polypeptide conjugates comprise one or more thioether bonds;
b) repeating (ii) up to 6 more times for each successive compound of claim 1 containing more than two stable isotopes;
(iii) combining the individual mixtures formed in (i) and (ii) to form a combined mixture;
(iv) enzymatically digesting the combined mixture to form a mixture of peptides comprising a combination of (a) one or more compounds of claim 1-polypeptide conjugates and (b) one or more isotopically labeled compounds of claim 1-polypeptide conjugates, whereby each conjugate is formed through one or more thioether bonds;
(v) capturing the polypeptide conjugates on a solid phase support;
(vi) contacting the thus-isolated polypeptide conjugates with a diboron reagent, thereby releasing the polypeptides;
(vii) analyzing the polypeptides via a targeted mass spectrometry assay;
(viii) detecting one or more thiolated ions, or derivatives ions thereof produced in the targeted mass spectrometry assay; and
(ix) determining target engagement stoichiometry for the compound of claim 1-polypeptide conjugate based on the ratio of thiolated ions, or derivative ions thereof, derived from the isotopically labeled to the unlabeled compounds of claim 1-peptide conjugates produced in the targeted mass spectrometry assay.

76-81. (canceled)

82. A method of quantifying the number of cysteine residues on a polypeptide that are targeted by a compound, comprising:
(i) reacting the compound of claim 1 at a fixed concentration with a first mixture comprising one or more polypeptides to form a second mixture comprising one or more compounds of claim 1-polypeptide conjugates, wherein each of the compounds of claim 1-polypeptide conjugates comprise one or more thioether bonds;
(ii) repeating (i)×3 to form a third, fourth, and fifth mixtures;
(iii) enzymatically digesting the second, third, fourth, and fifth mixtures;
(iv) capturing the polypeptides on a solid phase support;
(v) isotopically labeling the captured polypeptides from the third, fourth, and fifth mixtures;
(vi) combining the individually captured polypeptides to form a combined mixture of captured polypeptides;
(vi) contacting the captured polypeptides with a diboron reagent, thereby releasing the polypeptides;
(vii) analyzing the polypeptides via a targeted mass spectrometry assay;
(viii) detecting one or more thiolated ions, or derivatives ions thereof produced in the targeted mass spectrometry assay; and
(ix) determining target engagement stoichiometry for the compound of claim 1-polypeptide conjugate based on the ratio of thiolated ions, or derivative ions thereof, derived from the isotopically labeled to the unlabeled compounds of claim 1-peptide conjugates produced in the targeted mass spectrometry assay.
